(19) United States
(12) Patent Application Publication
(10) Pub. No.: US 2026/0062755 A1
(43) Pub. Date: Mar. 5, 2026

(54) PREDICTION AND MONITORING OF IMMUNOTHERAPY IN CANCER

(71) Applicant: InnoSIGN B.V., Eindhoven (NL)

(72) Inventors: Martijn Theodorus Lambert Akse, Eindhoven (NL); Paul Arnold Van De Wiel, Eindhoven (NL); Laurentius Henricus Franciscus Maria Holtzer, Eindhoven (NL); Anne Godefrida Catharina Van Brussel, Eindhoven (NL)

(73) Assignee: InnoSIGN B.V., Eindhoven (NL)

(21) Appl. No.: 19/102,702

(22) PCT Filed: Jul. 21, 2023
(86) PCT No.: PCT/EP2023/070354

§ 371 Date: Feb. 10, 2025

(30) Foreign Application Priority Data
Aug. 12, 2022 (EP) 22190224.0

(51) Int. Cl. C12Q 1/6886; A61K 45/00

(52) U.S. Cl. CPC C12Q 1/6886; A61K 45/00; C12Q 2600/106; C12Q 2600/158

(57) ABSTRACT

The invention relates to a method for predicting whether a subject with cancer can successfully be treated with an immunotherapy. The method is based on measuring the TGFbeta and/or the MAPK pathway activity in a sample obtained from the subject, wherein a low TGFbeta and/or MAPK pathway activity indicates that immunotherapy is likely successful and a high TGFbeta and/or MAPK pathway activity indicates that immunotherapy is not likely to be successful. The invention further relates to an immunotherapy for use in the treatment of cancer, the use comprising determining the TGFbeta and/or MAPK pathway activity and administering the immunotherapy if the treatment is deemed likely to succeed. The invention further relates to kits of parts and their uses in the methods described herein.

### FIELD OF THE INVENTION

The invention relates to methods of predicting a treatment outcome in a patient. Particularly the method relates to predicting immunotherapy outcome in cancer patients, based on the observed TGFbeta cellular signaling pathway activity in a sample from the patient. The invention further relates to immunotherapy for

### BACKGROUND OF THE INVENTION

Cancer remains one of the main causes of death in the developed world. Therefore there is a constant need for novel therapeutic strategies to further improve the treatment chances of patients suffering from cancer. One recent development is immunotherapy where the power of the body's immune system is harnessed to fight malignant cells.

As an example, Bacillus Calmette-Guerin (BCG) which is developed as a tuberculosis vaccine elicits an immune response, and is now commonly used in the treatment of early stage bladder cancer. More recent developments focus on immune checkpoint inhibitors. It was found that the immune system naturally is able to fight tumor cells, however tumors may evolve ways to actively block the immune system in order to evade it. By suppressing the tumor initiated local immune suppression, the patient's immune system can be “reactivated” to target tumor cells. Such treatments may be used by itself or in combination with other therapy, such as chemotherapy or radiation therapy, and are being used or tested for virtually any cancer type. Although this approach is very effective for patients who respond to the therapy, a relatively large subset of patients unfortunately does not respond to immunotherapy. Furthermore many immunotherapy approaches are very costly.

Therefore there is an unmet need for improved ways to stratify patients so that at an early stage those patients can be selected for which immunotherapy is likely to be beneficial, and to find alternative treatment options for those patients that are likely not to respond to immunotherapy.

For example, Ni et al. (NPJ Precision Oncology vol. 5, no. 1, 17-12-2021) describe a six gene TGFbeta related gene signature for predictive of resistance to immune checkpoint inhibitors in gynecological cancers. Further Mariathasan et al. (Nature 2018 Feb. 22; 554(7693):544-548) mention a pan-fibroblast TGFbeta response signature with low average expression in immune deserts but higher expression in inflamed and excluded tumors. However further and improved methods are needed to provide improved diagnosis options.

These issues, among others, are overcome by the methods, uses and products as defined in the appended claims.

### SUMMARY OF THE INVENTION

In a first aspect the invention relates to a method of predicting or monitoring a treatment response of a subject with cancer to immunotherapy, wherein the method is based on a sample obtained from the subject, the method comprising: determining the TGFbeta cellular signaling pathway activity in the sample, wherein an increased TGFbeta cellular signaling pathway activity correlates with a decreased probability of a favorable response to immunotherapy by the subject and wherein a decreased TGFbeta cellular signaling pathway activity correlates with an increased probability of a favorable response to immunotherapy by the subject, making a prediction about the treatment response based on the TGFbeta cellular signaling pathway activity, and wherein the TGFbeta cellular signaling pathway activity is determined based on the expression levels of three or more TGFbeta cellular signaling pathway target genes selected from: ANGPTL4, CDC42EP3, CDKN1A, CDKN2B, CCN2, GADD45A, GADD45B, HMGA2, ID1, IL11, INPP5D, JUNB, MMP2, MMP9, NKX2-5, OVOL1, PDGFB, PTHLH, SERPINE1, SGK1, SKIL, SMAD4, SMAD5, SMAD6, SMAD7, SNAI1, SNAI2, TIMP1, and VEGFA,
wherein the immunotherapy is an immune checkpoint inhibitor.

In a second aspect the invention relates to an immunotherapy for use in the treatment of cancer in a subject in need thereof, wherein the use comprises: determining the TGFbeta cellular signaling pathway activity in the sample, wherein an increased TGFbeta cellular signaling pathway activity correlates with a decreased probability of a favorable response to immunotherapy by the subject and wherein a decreased TGFbeta cellular signaling pathway activity correlates with an increased probability of a favorable response to immunotherapy by the subject, making a prediction about the treatment response based on the TGFbeta cellular signaling pathway activity, and wherein the TGFbeta cellular signaling pathway activity is determined based on the expression levels of three or more TGFbeta cellular signaling pathway target genes selected from: ANGPTL4, CDC42EP3, CDKN1A, CDKN2B, CCN2, GADD45A, GADD45B, HMGA2, ID1, IL11, INPP5D, JUNB, MMP2, MMP9, NKX2-5, OVOL1, PDGFB, PTHLH, SERPINE1, SGK1, SKIL, SMAD4, SMAD5, SMAD6, SMAD7, SNAI1, SNAI2, TIMP1, and VEGFA; and wherein the immunotherapy is an immune checkpoint inhibitor. In a third aspect the invention relates to the use of a kit of parts in predicting or monitoring a treatment response of a subject with cancer, the kit comprising:
means for determining the expression levels of three or more genes selected from: ANGPTL4, CDC42EP3, CDKN1A, CDKN2B, CCN2, GADD45A, GADD45B, HMGA2, ID1, IL11, INPP5D, JUNB, MMP2, MMP9, NKX2-5, OVOL1, PDGFB, PTHLH, SERPINE1, SGK1, SKIL, SMAD4, SMAD5, SMAD6, SMAD7, SNAI1, SNAI2, TIMP1, and VEGFA.

### BRIEF DESCRIPTION OF THE FIGURES

FIG. 1 depicts a graph showing TGFbeta pathway activities determined in RNA sequencing data from public dataset IMvigor210. The RNA sequencing data has been obtained from tumor biopsies from patients with bladder cancer prior to treatment with anti PD-L1 immune checkpoint inhibitors. Patients were followed up and scored based in treatment response, where CR=complete recovery, PR=partial recovery, SD=stable disease and PD=progressive disease. A clear distinction can be made between the complete/partial recovery group and the stable/progressive group (P=0.018) (left graph). Individual results are plotted in the right graph.

FIG. 2 depicts a graph showing MAPK pathway activities determined in RNA sequencing data from public dataset IMvigor210. The RNA sequencing data has been obtained from tumor biopsies from patients with bladder cancer prior to treatment with anti PD-L1 immune checkpoint inhibitors. Patients were followed up and scored based in treatment response, where CR=complete recovery, PR=partial recovery, SD=stable disease and PD=progressive disease. A clear distinction can be made between the complete/partial recovery group and the stable/progressive group (P=0.066) (left graph). Individual results are plotted in the right graph.

FIG. 3 depicts a graph showing TGFbetaR2 expression levels obtained from RNA sequencing data from public dataset IMvigor210. The RNA sequencing data has been obtained from tumor biopsies from patients with bladder cancer prior to treatment with anti PD-L1 immune checkpoint inhibitors. Patients were followed up and scored based in treatment response, where CR=complete recovery, PR=partial recovery, SD=stable disease and PD=progressive disease. No significant difference can be observed between the complete/partial recovery group and the stable/progressive group (P=0.23).

FIG. 4 depicts a graph showing TGFbeta (top graph) and MAPK (bottom graph) pathway activities determined in RNA sequencing data from public dataset GSE78220. The RNA sequencing data has been obtained from tumor biopsies from patients with melanoma prior to treatment with anti PD-1 immune checkpoint inhibitors. Patients were followed up and scored based in treatment response. Statistical analysis was performed using Mann Whitney test (T-test unpaired non-parametric): TGFb responders vs non-responders: P=0.0102; MAPK responders vs non-responders: P=0.0045.

FIG. 5 for each possible combination of 3, 4 or 5 genes selected from the full 29 gene set of TGFbeta target genes, pathway scores were calculated based on the full gene set. For each model the mean pathway activity score was calculated for the group of non-responders and for the group of responders (the same dataset as in FIG. 1 is used, public dataset IMvigor210). Then the difference of those 2 means was calculated. For all 3/4/5 gene models these differences are shown in a histogram.

FIGS. 6, 7 and 8 display box plots of 10 randomly selected 3, 4 or 5 gene models (selected from the full 29 gene TGFbeta target gene model). Pathway scores are not normalized to a 0-100 scale and that the possible range of scores depends on the genes in the model. FIG. 6 depicts 10 random 3 gene models, FIG. 7 depicts 10 random 4 gene models and FIG. 8 depicts 10 randomly selected 5 gene models.

FIG. 9 for each possible combination of 3, 4 or 5 genes selected from the 19 gene subset of TGFbeta target genes, pathway scores were calculated based on the subset. For each model the mean pathway activity score was calculated for the group of non-responders and for the group of responders (the same dataset as in FIG. 1 is used, public dataset IMvigor210). Then the difference of those 2 means was calculated. For all 3/4/5 gene models these differences are shown in a histogram.

FIGS. 10, 11 and 12 display box plots of 10 randomly selected 3, 4 or 5 gene models (selected from the 19 gene subset TGFbeta target gene model). Pathway scores are not normalized to a 0-100 scale and that the possible range of scores depends on the genes in the model. FIG. 10 depicts 10 random 3 gene models, FIG. 11 depicts 10 random 4 gene models and FIG. 12 depicts 10 randomly selected 5 gene models.

FIG. 13 for each possible combination of 3, 4 or 5 genes selected from the 10 gene subset of TGFbeta target genes, pathway scores were calculated based on the subset. For each model the mean pathway activity score was calculated for the group of non-responders and for the group of responders (the same dataset as in FIG. 1 is used, public dataset IMvigor210). Then the difference of those 2 means was calculated. For all 3/4/5 gene models these differences are shown in a histogram.

FIGS. 14, 15 and 16 display box plots of 10 randomly selected 3, 4 or 5 gene models (selected from the full 29 gene TGFbeta target gene model). Pathway scores are not normalized to a 0-100 scale and that the possible range of scores depends on the genes in the model. FIG. 14 depicts 10 random 3 gene models, FIG. 15 depicts 10 random 4 gene models and FIG. 16 depicts 10 randomly selected 5 gene models.

FIG. 17 For all possible 3 gene models TGFbeta pathway scores were calculated. The 3 genes were selected from the 10 gene set subset of target genes. For each model the mean pathway activity score was calculated for the group of responders and for the group of non-responders. Then the difference of those 2 means was calculated. For all 3 gene models these differences are shown in a histogram.

FIG. 18 displays box plots of 10 randomly selected 3 gene models (selected from the 10 gene subset of TGFbeta target genes). Pathway scores are not normalized to a 0-100 scale and the possible range of scores depends on the genes in the model.

FIG. 19 For all possible 3 gene models TGFbeta pathway scores were calculated. The 3 genes were selected from the 19 gene set subset of target genes. For each model the mean pathway activity score was calculated for the group of responders and for the group of non-responders. Then the difference of those 2 means was calculated. For all 3 gene models these differences are shown in a histogram.

FIG. 20 displays box plots of 10 randomly selected 3 gene models (selected from the 19 gene subset of TGFbeta target genes). Pathway scores are not normalized to a 0-100 scale and the possible range of scores depends on the genes in the model.

FIG. 21 depicts a plot of TGFbeta pathway activity obtained from highly purified T cells from treatment naïve CLL patients treated for 18 hours with avadomide or anti-PD-1 or anti-PD-L1 or combinations thereof. Groups were made representing poor or good prognosis.

### DEFINITIONS

For purposes of the present invention, the following terms are defined below.

As used herein, the singular form terms “A,” “an,” and “the” include plural referents unless the content clearly dictates otherwise. Thus, for example, reference to “a cell” includes a combination of two or more cells, and the like.

As used herein, the term “and/or” refers to a situation wherein one or more of the stated cases may occur, alone or in combination with at least one of the stated cases, up to with all of the stated cases.

As used herein, the term “antigen” refers to a substance to which a binding portion of an antibody may bind. The specific immunoreactive sites within the antigen are known as “epitopes” (or antigenic determinants). A target for an antibody, or antigen-binding portion thereof, may comprise an antigen, such as is defined herein.

As used herein, the term “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc. As used herein, the term “at most” a particular value means that particular value or less. For example, “at most 5” is understood to be the same as “5 or less” i.e., 5, 4, 3, . . . 10, −11, etc.

As used herein, the word “comprise” or variations thereof such as “comprises” or “comprising” will be understood to include a stated element, integer or step, or group of elements, integers or steps, but not to exclude any other element, integer or steps, or groups of elements, integers or steps. The verb “comprising” includes the verbs “essentially consisting of” and “consisting of”.

As used herein, the term “conventional techniques” refers to a situation wherein the methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al., Molecular Cloning. A Laboratory Manual, 2nd Edition, Cold Spring Harbor Laboratory Press, Cold Spring Harbor, N. Y., 1989; Ausubel et al., Current Protocols in Molecular Biology, John Wiley & Sons, New York, 1987 and periodic updates; and the series Methods in Enzymology, Academic Press, San Diego.

As used herein, the term “identity” refers to a measure of the identity of nucleotide sequences or amino acid sequences. In general, the sequences are aligned so that the highest order match is obtained. “Identity” per se has an art-recognized meaning and can be calculated using published techniques. See, e.g.: (Computational Molecular Biology, Lesk, A. M., ED., Oxford University Press, New York, 1988; Biocomputing: Informatics And Genome Projects, Smith, D. W., ED., Academic Press, New York, 1993; Computer Analysis Of Sequence Data, Part I, Griffin, A. M., And Griffin, H. G., EDS., Humana Press, New Jersey, 1994; Sequence Analysis In Molecular Biology, Von Heinje, G., Academic Press, 1987; and Sequence Analysis Primer; Gribskov, M. and Devereux, J., eds., M Stockton Press, New York, 1991). While there exist a number of methods to measure identity between two nucleotide sequences or amino acid sequences, the term “identity” is well known to skilled artisans (Carillo, H., and Lipton, D., SIAM J. Applied Math (1988) 48:1073). Methods commonly employed to determine identity or similarity between two sequences include, but are not limited to, those disclosed in Guide To Huge Computers, Martin J. Bishop, ed., Academic Press, San Diego, 1994, and Carillo, H., and Lipton, D., Siam J. Applied Math (1988) 48:1073. Methods to determine identity and similarity are codified in computer programs. Preferred computer program methods to determine identity and similarity between two sequences include, but are not limited to, GCG program package (Devereux, J., et al., Nucleic Acids Research (1984) 12(1):387), BLASTP, BLASTN, FASTA (Atschul, S. F. et al., J. Molec. Biol. (1990) 215:403).

As an illustration, by a polynucleotide having a nucleotide sequence having at least, for example, 95% “identity” to a reference nucleotide sequence encoding a polypeptide of a certain sequence, it is intended that the nucleotide sequence of the polynucleotide is identical to the reference sequence except that the polynucleotide sequence may include up to five point mutations per each 100 nucleotides of the reference amino acid sequence. In other words, to obtain a polynucleotide having a nucleotide sequence at least 95% identical to a reference nucleotide sequence, up to 5% of the nucleotides in the reference sequence may be deleted and/or substituted with another nucleotide, and/or a number of nucleotides up to 5% of the total nucleotides in the reference sequence may be inserted into the reference sequence. These mutations of the reference sequence may occur at the 5′ or 3′ terminal positions of the reference nucleotide sequence, or anywhere between those terminal positions, interspersed either individually among nucleotides in the reference sequence or in one or more contiguous groups within the reference sequence.

Similarly, by a polypeptide having an amino acid sequence having at least, for example, 95% “identity” to a reference amino acid sequence of SEQ ID NO: X is intended that the amino acid sequence of the polypeptide is identical to the reference sequence except that the amino acid sequence may include up to five amino acid alterations per each 100 amino acids of the reference amino acid of SEQ ID NO: X. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As used herein, the term “in vitro” refers to experimentation or measurements conducted using components of an organism that have been isolated from their natural conditions.

As used herein, the term “ex vivo” refers to experimentation or measurements done in or on tissue from an organism in an external environment with minimal alteration of natural condition.

As used herein, the term “nucleic acid”, “nucleic acid molecule” and “polynucleotide” is intended to include DNA molecules and RNA molecules. A nucleic acid (molecule) may be single-stranded or double-stranded, but preferably is double-stranded DNA.

As used herein, the terms “sequence” when referring to nucleotides, or “nucleic acid sequence”, “nucleotide sequence” or “polynucleotide sequence” refer to the order of nucleotides of, or within, a nucleic acid and/or polynucleotide. Within the context of the current invention a first nucleic acid sequence may be comprised within or overlap with a further nucleic acid sequence.

As used herein, the term “subject” or “individual” or “animal” or “patient” or “mammal,” used interchangeably, refer to any subject, particularly a mammalian subject, for whom diagnosis, prognosis, or therapy is desired. Mammalian subjects include humans, domestic animals, farm animals, and zoo-, sports-, or pet-animals such as dogs, cats, guinea pigs, rabbits, rats, mice, horses, cattle, cows, bears, and so on. As defined herein a subject may be alive or dead. Samples can be taken from a subject post-mortem, i.e. after death, and/or samples can be taken from a living subject.

As used herein, terms “treatment”, “treating”, “palliating”, “alleviating” or “ameliorating”, used interchangeably, refer to an approach for obtaining beneficial or desired results including, but not limited to, therapeutic benefit. By therapeutic benefit is meant eradication or amelioration or reduction (or delay) of progress of the underlying disease being treated. Also, a therapeutic benefit is achieved with the eradication or amelioration or reduction (or delay) of progress of one or more of the physiological symptoms associated with the underlying disease such that an improvement or slowing down or reduction of decline is observed in the patient, notwithstanding that the patient can still be afflicted with the underlying disease.

### DETAILED DESCRIPTION OF THE INVENTION

The section headings as used herein are for organizational purposes only and are not to be construed as limiting the subject matter described.

A portion of this invention contains material that is subject to copyright protection (such as, but not limited to, diagrams, device photographs, or any other aspects of this submission for which copyright protection is or may be available in any jurisdiction). The copyright owner has no objection to the facsimile reproduction by anyone of the patent document or patent invention, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention relates, unless otherwise indicated. Other specifically defined terms are to be construed in a manner consistent with the definition as provided herein. The preferred materials and methods are described herein, although any methods and materials similar or equivalent to those described herein can be used in the practice for testing of the present invention.

Unless otherwise defined, all technical and scientific terms used herein have the same meaning as commonly understood by a person of ordinary skill in the art.

Treatment of patients with cancer using immunotherapy has resulted in remarkable recovery in some cases, however the percentage of patients which do not show a response to such treatment is unfortunately very high. For example depending on the cancer type, 75% or more of the patients may be non-responders. This is a problem for several reasons. First of all for the patients valuable time is lost before it is found out that the immunotherapy does not work and the patients can be switched to alternative treatment methods. By the time it is found out that the immunotherapy is not working, it may already be too late to start alternative treatment options. Furthermore, most immunotherapy treatments are very costly, therefore treatment with such is preferably avoided if it is clear the treatment will (most likely) not work. Unfortunately there are currently no good ways to predict whether immunotherapy will work in a patient with cancer.

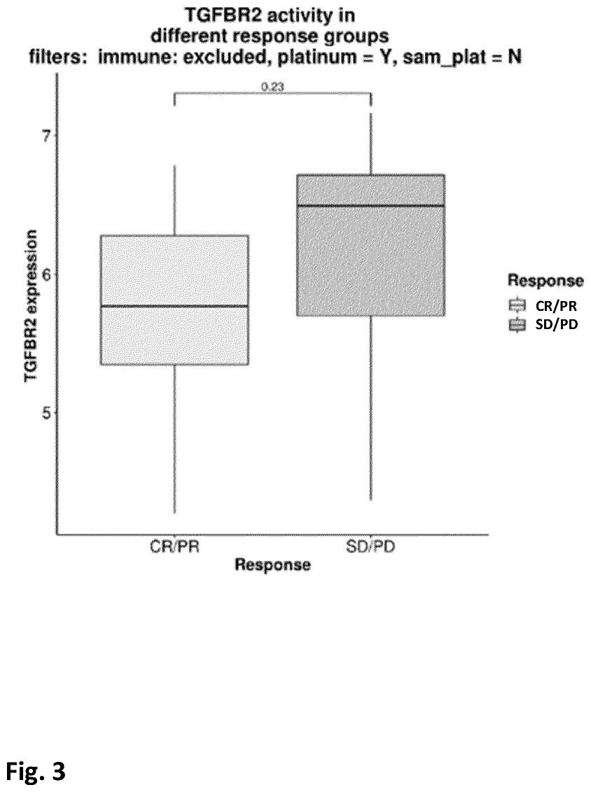

Recently more evidence is accumulating that the TGFbeta cellular signaling pathway may play a role in tumor evasion of the immune system. For example, Mariathasan et al. (Nature. 2018 Feb. 22; 554(7693):544-548) describe that excessive TGFbeta signaling may contribute to exclusion of T cells from the tumor environment, thereby preventing an immune response towards the tumor. It has further been suggested that blocking TGFbeta signaling combined with immunotherapy may be a viable strategy for treating non-responders to immunotherapy. It is however challenging to predict which patients will respond to immunotherapy. Currently no reliable tests are available. Further, the applicant's data presented here (see FIG. 3 and corresponding text in the Examples) demonstrates that expression of TGFBR2 does not significantly distinguish between responders and non-responders to immunotherapy. Therefore, even though the role of TGFbeta in tumor cells evading the immune system has been described, this has not resulted in reliable methods for predicting a response to immunotherapy treatment in a cancer patient. The inventors here for the first time show a method for making such prediction, among others, in a reliable manner. The same holds true for the MAPK pathway which is also described herein.

Therefore, in a first aspect the invention relates to a method of predicting or monitoring a treatment response of a subject with cancer to immunotherapy, wherein the method is based on a sample obtained from the subject, the method comprising:
determining the TGFbeta cellular signaling pathway activity in the sample,
wherein an increased TGFbeta cellular signaling pathway activity correlates with a decreased probability of a favorable response to immunotherapy by the subject and wherein a decreased TGFbeta cellular signaling pathway activity correlates with an increased probability of a favorable response to immunotherapy by the subject,
making a prediction about the treatment response based on the TGFbeta cellular signaling pathway activity, and
wherein the TGFbeta cellular signaling pathway activity is determined based on the expression levels of three or more, such as three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight or twenty-nine, TGFbeta cellular signaling pathway target genes selected from: ANGPTL4, CDC42EP3, CDKN1A, CDKN2B, CCN2, GADD45A, GADD45B, HMGA2, ID1, IL11, INPP5D, JUNB, MMP2, MMP9, NKX2-5, OVOL1, PDGFB, PTHLH, SERPINE1, SGK1, SKIL, SMAD4, SMAD5, SMAD6, SMAD7, SNAI1, SNAI2, TIMP1, and VEGFA,
wherein the immunotherapy is an immune checkpoint inhibitor.

Also disclosed herein is a method of predicting or monitoring a treatment response of a subject with cancer to immunotherapy, wherein the method is based on a sample obtained from the subject, the method comprising:
determining the TGFbeta cellular signaling pathway activity and/or the MAPK-AP1 cellular signaling pathway activity in the sample, wherein an increased TGFbeta and/or the MAPK-AP1 cellular signaling pathway activity correlates with a decreased probability of a favorable response to immunotherapy by the subject and wherein a decreased TGFbeta and/or MAPK-AP1 cellular signaling pathway activity correlates with an increased probability of a favorable response to immunotherapy by the subject, making a prediction about the treatment response based on the TGFbeta cellular signaling pathway activity and/or the MAPK-AP1 cellular signaling pathway activity, and wherein the TGFbeta cellular signaling pathway activity is determined based on the expression levels of three or more TGFbeta cellular signaling pathway target genes selected from: ANGPTL4, CDC42EP3, CDKN1A, CDKN2B, CCN2, GADD45A, GADD45B, HMGA2, ID1, IL11, INPP5D, JUNB, MMP2, MMP9, NKX2-5, OVOL1, PDGFB, PTHLH, SERPINE1, SGK1, SKIL, SMAD4, SMAD5, SMAD6, SMAD7, SNAI1, SNAI2, TIMP1, and VEGFA, wherein the MAPK-AP1 cellular signaling pathway activity is determined based on the expression levels of three or more AP1 target genes selected from: BCL2L11, CCND1, DDIT3, DNMT1, EGFR, ENPP2, EZR, FASLG, FIGF, GLRX, IL2, IVL, LOR, MMP1, MMP3, MMP9, SERPINE1, PLAU, PLAUR, PTGS2, SNCG, TIMP1, TP53, and VIM, and wherein the immunotherapy is an immune checkpoint inhibitor.

Further disclosed herein is method of predicting or monitoring a treatment response of a subject with cancer to immunotherapy, wherein the method is based on a sample obtained from the subject, the method comprising: determining the TGFbeta cellular signaling pathway activity in the sample, wherein an increased TGFbeta cellular signaling pathway activity correlates with a decreased probability of a favorable response to immunotherapy by the subject and wherein a decreased TGFbeta cellular signaling pathway activity correlates with an increased probability of a favorable response to immunotherapy by the subject, making a prediction about the treatment response based on the TGFbeta cellular signaling pathway activity, and wherein the TGFbeta cellular signaling pathway activity is determined based on the expression levels of three or more TGFbeta cellular signaling pathway target genes selected from: ANGPTL4, CDC42EP3, CDKN1A, CDKN2B, CCN2, GADD45A, GADD45B, HMGA2, ID1, IL11, INPP5D, JUNB, MMP2, MMP9, NKX2-5, OVOL1, PDGFB, PTHLH, SERPINE1, SGK1, SKIL, SMAD4, SMAD5, SMAD6, SMAD7, SNAI1, SNAI2, TIMP1, and VEGFA.

Further disclosed herein is a method of predicting or monitoring a treatment response of a subject with cancer to immunotherapy, wherein the method is based on a sample obtained from the subject, the method comprising: determining the MAPK-AP1 cellular signaling pathway activity in the sample, wherein an increased MAPK-AP1 cellular signaling pathway activity correlates with a decreased probability of a favorable response to immunotherapy by the subject and wherein a decreased MAPK-AP1 cellular signaling pathway activity correlates with an increased probability of a favorable response to immunotherapy by the subject, making a prediction about the treatment response based on the MAPK-AP1 cellular signaling pathway activity, and wherein the MAPK-AP1 cellular signaling pathway activity is determined based on the expression levels of three or more AP1 target genes selected from: BCL2L11, CCND1, DDIT3, DNMT1, EGFR, ENPP2, EZR, FASLG, FIGF, GLRX, IL2, IVL, LOR, MMP1, MMP3, MMP9, SERPINE1, PLAU, PLAUR, PTGS2, SNCG, TIMP1, TP53, and VIM.

The present invention revolves around the finding that, pretreatment, in a sample obtained from a subject TGFbeta and/or MAPK pathway activity can be determined using the methods described herein, and that TGFbeta and MAPK pathway activities strongly correlates with a treatment response to immunotherapy, as demonstrated in the Examples below. A treatment response when used herein generally refers to the response of a subject with cancer to immunotherapy. Such response may be a favorable response or a non-favorable response. Therefore the subjects are also referred to as responders or non-responders respectively. Unless from the context it appears otherwise the term “a response” when referring to treatment with immunotherapy is intended to mean a favorable response. Thus, in an embodiment the treatment response of the subject is favorable or non-favorable response to the immunotherapy treatment. For example treatment responses can be divided in complete response (CP), partial response (PR), stable disease (SD) or disease progression (DP) as described in Eisenhauer et al (European Journal of Cancer 45 (2009) 228-247; hereby incorporated by reference in its entirety). Generally complete and partial response will be evaluated as a favorable outcome, while disease progression is evaluated as a non-favorable response to treatment with an immunotherapy. Stable disease, may depending on the context of the disease and the progression be evaluated as favorable or non-favorable. Therefore in an embodiment the response to immunotherapy is complete response, partial response, stable disease or disease progression.

When used herein the term cancer may refer to any type of cancer. The data presented herein demonstrates a predictive effect in metastatic urothelial cancer and in melanoma as well as T cells isolated from chronic lymphocytic leukemia patients, however it is appreciated that the underlying principle preventing activation of the PD-1 receptor on a T-cell, allowing the T-cell mediated destruction of the a tumor cell is likely to apply to any type of cancer. That is, TGFbeta pathway activity appears to drive resistance to immune checkpoint inhibitors regardless of the tumor type. Thus by presenting data from the two tumor types presented here the applicant has made it plausible that the underlying predictive effect can be applied generally. Thus in an embodiment the invention relates to a method of predicting or monitoring a treatment response of a subject with cancer to immunotherapy, wherein the cancer is a cancer type that is typically treated with an immune checkpoint inhibitor, however it is appreciated that the method may actually also be interesting for cancer types that typically not treated with immune checkpoint inhibitors due to the low response rates, as the present method may identify those responders (by having low TGFbeta and/or MAPK pathway activity).

Examples of cancers that may be treated with immune checkpoint inhibitors include breast cancer, bladder cancer, cervical cancer, colon cancer, head and neck cancer, Hodgkin lymphoma, liver cancer, lung cancer, renal cell cancer, skin cancer (including melanoma), stomach cancer, rectal cancer, or any solid tumor that is not able to repair errors in its DNA that occur when the DNA is copied. Thus in an embodiment the invention relates to a method of predicting or monitoring a treatment response of a subject with cancer to immunotherapy, wherein the cancer is breast cancer, bladder cancer, cervical cancer, colon cancer, head and neck cancer, Hodgkin lymphoma, liver cancer, lung cancer, renal cell cancer, skin cancer (including melanoma), stomach cancer, rectal cancer, or any solid tumor that is not able to repair errors in its DNA that occur when the DNA is copied. In a particularly preferred embodiment the cancer is bladder cancer, melanoma or leukemia, particularly metastatic bladder cancer or metastatic melanoma or chronic lymphocytic leukemia. The method used herein may be used for predicting or monitoring. When used herein, predicting refers to the situation wherein the TGFbeta and/or the MAPK pathway is determined in a sample obtained from a subject, and wherein the sample is obtained prior to onset of treatment with immunotherapy. When used herein, monitoring refers to the situation wherein the TGFbeta and/or MAPK pathway is determined in a sample obtained from a subject, and wherein the sample is obtained after onset of treatment with immunotherapy. It is understood that in the case of predicting, the TGFbeta and/or MAPK pathway activity is determined in a sample obtained prior to treatment with an immunotherapy and may be used to guide a treatment decision, e.g. whether to treat the subject with immunotherapy or with an alternative treatment. In the case of monitoring, the TGFbeta and/or MAPK pathway activity is determined in a sample obtained after onset of treatment with an immunotherapy and may be used to guide a decision whether to continue with existing treatment, e.g. whether to continue to treat the subject with immunotherapy or whether to change the subject to an alternative treatment. Therefore in an embodiment the method is for predicting a treatment response, wherein the method further comprises providing a treatment advice to a medical care giver, preferably wherein the treatment advice is to provide immunotherapy or to provide an alternative therapy. In an alternative embodiment the method is for monitoring a treatment response, wherein the method further comprises providing a continuation advice to a medical care giver, preferably wherein the continuation advice is to continue the current treatment strategy or to change the treatment strategy. It is understood that when a good outcome is predicted immunotherapy is recommended to the subject, and when a poor outcome is predicted an alternative treatment is recommended to the subject.

When used herein the term “alternative treatment” refers to a situation where the method predicts that immunotherapy is likely not successful and thus an alternative treatment should be sought for the patient. Available and suitable treatment strategies depend on the type of cancer and stage and are known to the skilled person, for example the treating physician. For example suitable treatment strategies comprise radiation therapy, chemotherapy, surgical resection. Alternative treatment may also refer to treatment of an immunotherapy in combination with a TGFbeta pathway inhibitor, treatment of an immunotherapy in combination with a MAPK pathway inhibitor, or treatment of an immunotherapy in combination with a TGFbeta pathway inhibitor and a MAPK pathway inhibitor.

When used herein a TGFbeta inhibitor may for example be a TGFbeta or TGFbeta receptor specific antibody, a ligand trap, a vaccine, a small molecule inhibitor such as a kinase inhibitor with specificity towards the TGFbeta receptor or an antisense oligonucleotide targeting TGFbeta or TGFbeta receptor encoding mRNA. Non limiting examples are listed in Huang et al., Recent progress in TGF-β inhibitors for cancer therapy, Biomedicine & Pharmacotherapy, Volume 134, 2021, 111046, hereby incorporated by reference in its entirety.

When used herein a MAPK inhibitor may for example be a growth factor or growth factor receptor specific antibody, a ligand trap, a vaccine, a small molecule inhibitor such as a kinase inhibitor with specificity towards the growth factor receptor or one of its downstream components such as but not limited to MAPK, MAPKK or MAPKKK, or an antisense oligonucleotide targeting a growth factor or a growth factor receptor encoding mRNA, or mRNA encoding one of the downstream components of the MAPK pathway such as but not limited to MAPK, MAPKK or MAPKKK. Non limiting examples are listed in Burkhard K, Shapiro P. Use of inhibitors in the study of MAP kinases. Methods Mol Biol. 2010; 661:107-22, hereby incorporated by reference in its entirety.

The present invention is based on a method of determining the TGFbeta cellular signaling pathway activity in a sample obtained from a subject. The sample may for example be a tumor biopsy, including liquid biopsies. Therefore in an embodiment the sample is a tissue sample, a blood sample, a biopsy, or a sample from the subject comprising cells. It is understood that the method does not necessarily include the step of obtaining a biopsy of a subject. Therefore in an embodiment the method includes the step of providing a previously obtained sample form a subject. Alternatively the method may also include a step of obtaining a tumor biopsy from the subject with cancer. As shown herein below in the examples, TGFbeta activity in either the tumor cells or T cells may be predictive for treatment response. Therefore in a further embodiment the sample preferably includes a tumor cell and/or a T cell. The tumor cell may be a cell obtained from the primary tumor, a metastasis or a circulating tumor cell.

A sample when used herein refers to a sample obtained from a subject comprising at least cells. Preferably the cells in the sample are either cells that may secrete TGFbeta, or cells on which secreted TGFbeta may exert an effect, such as for example tumor cells or immune cells. It is understood however that the TGFbeta signaling may also effect surrounding tissues or even more distant cells such as blood cells, therefore it is not required that the sample comprises tumor cells or immune cells. Additionally the cells may be cells with a MAPK pathway activity that may be influenced by tumor immune evasion responses.

In the present method, the TGFbeta cellular signaling pathway activity may be determined in a sample. The terms TGFbeta, TGFb, TGF β and TGF-β are used interchangeably and refer to Transforming Growth Factor beta. Further the terms pathway activity, signaling pathway activity and cellular signaling pathway are used interchangeably. TGFbeta cellular signaling pathway activity, when used herein, refers to actions caused by the signaling cascade initiated by binding of TGFbeta to its receptor. The downstream signaling generally results in changes in expression (up- or downregulation) of target genes of the pathway. TGFbeta may refer to any of TGFB1, TGFb2 or TGFB3, which are ligands capable of binding to the TGFbeta receptor to initiate the pathway. Generally the ligands will bind to a type II TGFbeta receptor, typically TGFbetaR2, which further complexes with type 1 receptors such as ALK1 or ALK5 (also known as TGFbetaR1), and acts downstream through SMAD family proteins, typically SMAD2, SMAD3 and SMAD4 as for example described in Wrana et al. (Cell vol. 71, Issue 6, pages 1003-1014, 1992; hereby incorporated by reference in its entirety)

In accordance with a main aspect of the present invention, the above problem is solved by a method for inferring activity of a TGFbeta cellular signaling pathway in a sample obtained from a subject. The method may be a computer-implemented performed by a digital processing device. In a preferred embodiment the inferring comprises: determining or receiving expression levels of three or more, for example, three, four, five, six, seven, eight, nine, ten, eleven, twelve or more, target genes of the TGFbeta cellular signaling pathway measured in a sample of the subject, determining an activity level of a TGFbeta transcription factor (TF) element in the sample of the subject, the TGFbeta TF element controlling transcription of the three or more TGF β target genes, the determining being based on evaluating a calibrated mathematical model pathway relating the expression levels of the three one or more TGFbeta target genes to the activity level of the TGFbeta TF element; inferring the activity of the TGFbeta cellular signaling pathway in the subject based on the determined activity level of the TGFbeta TF element in the sample of the subject, wherein the three or more TGF β target genes are selected from the group consisting of: ANGPTL4, CDC42EP3, CDKN1A, CDKN2B, CCN2, GADD45A, GADD45B, HMGA2, ID1, IL11, SERPINE1, INPP5D, JUNB, MMP2, MMP9, NKX2-5, OVOL1, PDGFB, PTHLH, SGK1, SKIL, SMAD4, SMAD5, SMAD6, SMAD7, SNAI1, SNAI2, TIMP1, and VEGFA. In a preferred embodiment the three or more target genes are selected from the group consisting of: ANGPTL4, CDC42EP3, CDKN1A, CCN2, GADD45A, GADD45B, HMGA2, ID1, IL11, JUNB, PDGFB, PTHLH, SERPINE1, SGK1, SKIL, SMAD4, SMAD5, SMAD6, SMAD7, SNAI2, VEGFA, more preferably, from the group consisting of: ANGPTL4, CDC42EP3, CDKN1A, CCN2, GADD45B, ID1, IL11, JUNB, SERPINE1, PDGFB, SKIL, SMAD7, SNAI2, and VEGFA, more preferably, from the group consisting of: ANGPTL4, CDC42EP3, ID1, IL11, JUNB, SERPINE1, SKIL, and SMAD7. Herein, the “activity level” of a TF element denotes the level of activity of the TF element regarding transcription of its target genes.

The present invention is based on the finding of the inventors that a suitable way of identifying effects occurring in the TGFbeta cellular signaling pathway can be based on a measurement of the signaling output of the TGFbeta cellular signaling pathway, which is—amongst others—the transcription of the target genes, which is controlled by a TGFbeta transcription factor (TF) element that is controlled by the TGFbeta cellular signaling pathway. This innovation by the inventors assumes that the TF activity level is at a quasi-steady state in the sample which can be detected by means of—amongst others—the expression values of the TGFbeta target genes. The TGFbeta cellular signaling pathway targeted herein is known to control many functions in many cell types in humans, such as proliferation, differentiation and wound healing. Regarding pathological disorders, such as cancer (e.g., colon, pancreatic, lung, brain or breast cancer), the TGFbeta cellular signaling pathway plays two opposite roles, either as a tumor suppressor or as a tumor promoter, which is detectable in the expression profiles of the target genes and thus exploited by means of a calibrated mathematical pathway model.

The present invention makes it possible to determine the activity of the TGFbeta cellular signaling pathway in a subject by (i) determining an activity level of a TGFbeta TF element in the sample of the subject, wherein the determining is based on evaluating a calibrated mathematical model relating the expression levels of three or more target genes of the TGFbeta cellular signaling pathway, the transcription of which is controlled by the TGFbeta TF element, to the activity level of the TGFbeta TF element, and by (ii) inferring the activity of the TGFbeta cellular signaling pathway in the subject based on the determined activity level of the TGFbeta TF element in the sample of the subject. This preferably allows improving the possibilities of characterizing patients that have a cancer, e.g., a colon, pancreatic, lung, brain or breast cancer, which have or have developed immunotherapy resistance that is at least partially driven by activity of the TGFbeta cellular signaling pathway, and that are therefore not likely to respond to immunotherapy, such as immune checkpoint inhibitors. In particular embodiments, treatment determination can be based on a specific TGFbeta cellular signaling pathway activity. In a particular embodiment, the TGFbeta cellular signaling status can be set at a threshold value of the TGFbeta cellular signaling pathway activity.

Herein, the term “TGFbeta transcription factor element” or “TGFbeta TF element” or “TF element” is defined to be a protein complex containing at least one or, preferably, a dimer of the TGFbeta members (SMAD1, SMAD2, SMAD3, SMAD5 and SMAD8 with SMAD4) or a trimer (two proteins from SMAD1, SMAD2, SMAD3, SMAD5 and SMAD8 with SMAD4), which is capable of binding to specific DNA sequences, thereby controlling transcription of target genes. Preferably, the term refers to either a protein or protein complex transcriptional factor triggered by the binding of TGFbeta to its receptor or an intermediate downstream signaling agent between the binding of TGFbeta to its receptor and the final transcriptional factor protein or protein complex. For example, it is known that TGFbeta binds to an extracellular TGFbeta receptor that initiates an intracellular “SMAD” signaling pathway and that one or more SMAD proteins (receptor-regulated or R-SMADs (SMAD1, SMAD2, SMAD 3, SMAD5 and SMAD8) and SMAD4) participate in, and may form a heterocomplex which participates in, the TGFbeta transcription signaling cascade which controls expression.

The calibrated mathematical pathway model may be a probabilistic model, preferably a Bayesian network model, based on conditional probabilities relating the activity level of the TGFbeta TF element and the expression levels of the three or more TGFbeta target genes, or the calibrated mathematical pathway model may be based on one or more linear combination(s) of the expression levels of the three or more TGFbeta target genes. In particular, the inferring of the activity of the TGFbeta cellular signaling pathway may be performed as disclosed in the published international patent application WO 2013/011479 A2 (“Assessment of cellular signaling pathway activity using probabilistic modeling of target gene expression”; hereby incorporated by reference in its entirety) or as described in the published international patent application WO 2014/102668 A2 (“Assessment of cellular signaling pathway activity using linear combination(s) of target gene expressions”; hereby incorporated by reference in its entirety), the contents of which are herewith incorporated in their entirety. Further details regarding the inferring of cellular signaling pathway activity using mathematical modeling of target gene expression can be found in Verhaegh W. et al., “Selection of personalized patient therapy through the use of knowledge-based computational models that identify tumor-driving signal transduction pathways”, Cancer Research, Vol. 74, No. 11, 2014, pages 2936 to 2945.

The term “target gene” as used herein, means a gene whose transcription is directly or indirectly controlled by a TGFbeta transcription factor element. The “target gene” may be a “direct target gene” and/or an “indirect target gene” (as described herein). Moreover, the “target gene(s)” may be “direct target genes” and/or “indirect target genes” (as described herein).

It is preferred that the three or more, for example, three, four, five, six, seven, eight, nine, ten, eleven, twelve or more, TGFbeta target genes are selected from the group consisting of: ANGPTL4, CDC42EP3, CDKN1A, CCN2, GADD45A, GADD45B, HMGA2, ID1, IL11, JUNB, PDGFB, PTHLH, SGK1, SKIL, SMAD4, SMAD5, SMAD6, SMAD7, SNAI2, VEGFA, more preferably, from the group consisting of: ANGPTL4, CDC42EP3, CDKN1A, CCN2, GADD45A, GADD45B, HMGA2, ID1, JUNB, PDGFB, PTHLH, SERPINE1, SGK1, SKIL, SMAD4, SMAD5, SMAD6, SMAD7, SNAI2, VEGFA.

It is further preferred that the three or more, for example, three, four, five, six, seven, eight, nine, ten, eleven, twelve or more, TGFbeta target genes are selected from the group consisting of: ANGPTL4, CDC42EP3, CDKN1A, CCN2, GADD45B, ID1, IL11, JUNB, PDGFB, SKIL, SMAD7, and SNAI2, more preferably, from the group consisting of: ANGPTL4, CDC42EP3, CDKN1A, CCN2, GADD45B, ID1, JUNB, SERPINE1, SKIL, SMAD7, SNAI2, and VEGFA.

It is further preferred that the three or more, for example, three, four, five, six, seven or more, TGFbeta target genes are selected from the group consisting of: ANGPTL4, CDC42EP3, ID1, IL11, JUNB, SKIL, and SMAD7, more preferably, from the group consisting of: ANGPTL4, CDC42EP3, ID1, JUNB, SERPINE1, SKIL, and SMAD7.

Preferably, the selected three or more TGFbeta target genes include ANGPTL4 and CDC42EP3, more preferably, ANGPTL4, CDC42EP3, ID1, JUNB, SERPINE1, SKIL, and SMAD7.

It is particularly preferred that the three or more TGF β target genes are ANGPTL4, CDC42EP3, CDKN1A, CCN2, GADD45B, ID1, JUNB, SERPINE1, SKIL, SMAD7, SNAI2, and VEGFA.

Additional details for modelling TGFbeta cellular signaling pathway activity in a subject are described in WO 2016/062891 (“ASSESSMENT OF TGF-β CELLULAR SIGNALING PATHWAY ACTIVITY USING MATHEMATICAL MODELLING OF TARGET GENE EXPRESSION”; hereby incorporated by reference in its entirety).

Another aspect of the present invention relates to a method (as described herein), further comprising:
determining whether the TGFbeta cellular signaling pathway is operating as a tumor promoter in the subject based on the inferred activity of the TGF β cellular signaling pathway in the subject.

In the present method, the MAPK cellular signaling pathway activity may be determined in a sample. The terms MAPK and MAPK-AP1 are used interchangeably herein and refer to a receptor/ligand initiated pathway that exerts its effect through the MAPK signaling cascade and the AP1 transcription complex. Activator protein 1 (AP-1) is a transcription factor that regulates gene expression in response to a variety of stimuli, including cytokines, growth factors, stress, and bacterial and viral infections. AP-1 controls a number of cellular processes including differentiation, proliferation, and apoptosis. The structure of AP-1 is a heterodimer composed of proteins belonging to the c-Fos, c-Jun, ATF and JDP families.

Herein, the term “AP1 transcription factor element” or “AP1 TF element” or “TF element” is defined to be a protein complex containing at least a member of the Jun (e.g. c-Jun, JunB and JunB) family and/or a member of the Fos (e.g. c-Fos, FosB, Fra-I and Fra-2) family and/or a member of the ATF family and/or a member of the JDP family, forming e.g. Jun˜June or Jun˜Fos dimers, capable of binding to specific DNA sequences, preferably the response elements 12-O-Tetradecanoylphorbol-13-acetate (TPA) response element (TRE) with binding motif or cyclic AMP response element (CRE) with binding motif, thereby controlling transcription of target genes.

Preferably, the term refers to either a protein or protein complex transcriptional factor triggered by the binding of AP1 inducing ligands, such as growth factors (e.g., EGF) and cytokines, to its receptor or an intermediate downstream signaling agent, or triggered by the presence of an AP1-activating mutation.

The calibrated mathematical pathway model may be a probabilistic model, preferably a Bayesian network model, based on conditional probabilities relating the activity level of the AP1 TF element and the expression levels of the three or more AP1 target genes, or the calibrated mathematical pathway model may be based on one or more linear combination(s) of the expression levels of the three or more AP1 target genes. In particular, the inferring of the activity of the MAPK-AP1 cellular signaling pathway may be performed as disclosed in the published international patent application WO 2013/011479 A2 and WO 2019/120658, both incorporated by reference in their entirety.

It is preferred that the three or more, for example, three, four, five, six, seven, eight, nine, ten, eleven, twelve or more, MAPK-AP1 target genes are selected from the group consisting of: BCL2L11, CCND1, DDIT3, DNMT1, EGFR, ENPP2, EZR, FASLG, FIGF, GLRX, IL2, IVL, LOR, MMP1, MMP3, MMP9, SERPINE1, PLAU, PLAUR, PTGS2, SNCG, TIMP1, TP53, and VIM, preferably, from the group consisting of: CCND1, EGFR, EZR, GLRX, MMP1, MMP3, PLAU, PLAUR, SERPINE1, SNCG, and TIMP1.

Using the methods described above a numeric value can be assigned to the TGFbeta cellular signaling pathway. For example, a value between 0 to 100 may be chosen to reflect no pathway activity (0) or maximum pathway activity (100). Alternatively intermediate pathway activity may be chosen as having a value 0, where negative values represent low pathway activity and thus high pathway activity is reflected by positive values. The assigned values allow comparison with other values, e.g. obtained from reference samples.

Therefore in an embodiment the method further comprises comparing the TGFbeta cellular signaling pathway activity and/or the MAPK-AP1 cellular signaling pathway activity with a predetermined threshold, and wherein the prediction is based on the comparison of the TGFbeta cellular signaling pathway activity and/or the MAPK-AP1 cellular signaling pathway activity with the predetermined threshold. In an embodiment the method further comprises comparing the TGFbeta cellular signaling pathway activity with a predetermined threshold, and wherein the prediction is based on the comparison of the TGFbeta cellular signaling pathway activity with the predetermined threshold. In an embodiment the method further comprises comparing the MAPK-AP1 cellular signaling pathway activity with a predetermined threshold, and wherein the prediction is based on the comparison of the MAPK-AP1 cellular signaling pathway activity with the predetermined threshold.

In a further embodiment the method comprises receiving expression levels of three or more target genes of the TGFbeta cellular signaling pathway and/or the MAPK-AP1 cellular signaling pathway measured in a sample of the subject, preferably wherein the method is a computer implemented method, or wherein the method comprises determining expression levels of three or more target genes of the TGFbeta cellular signaling pathway and/or the MAPK-AP1 cellular signaling pathway in a sample obtained from the subject. In an embodiment the method comprises receiving expression levels of three or more target genes of the TGFbeta cellular signaling pathway measured in a sample of the subject, preferably wherein the method is a computer implemented method, or wherein the method comprises determining expression levels of three or more target genes of the TGFbeta cellular signaling pathway in a sample obtained from the subject. In an embodiment the method comprises receiving expression levels of three or more target genes of the MAPK-AP1 cellular signaling pathway measured in a sample of the subject, preferably wherein the method is a computer implemented method, or wherein the method comprises determining expression levels of three or more target genes of the MAPK-AP1 cellular signaling pathway in a sample obtained from the subject.

In an embodiment the determining of the TGFbeta cellular signaling pathway activity and/or the MAPK-AP1 cellular signaling pathway activity comprises determining an activity level of a TGFbeta and/or MAPK-AP1 transcription factor (TF) element in the sample of the subject, the TGFbeta and/or the MAPK-AP1 TF element controlling transcription of the three or more TGFbeta and/or MAPK-AP1 cellular signaling pathway target genes, the determining being based on evaluating a calibrated mathematical pathway model relating the expression levels of the three or more TGFbeta and/or MAPK-AP1 target genes to the activity level of the TGFbeta and/or MAPK-AP1 TF element. In an embodiment the determining of the TGFbeta cellular signaling pathway activity comprises determining an activity level of a TGFbeta transcription factor (TF) element in the sample of the subject, the TGFbeta TF element controlling transcription of the three or more TGFbeta cellular signaling pathway target genes, the determining being based on evaluating a calibrated mathematical pathway model relating the expression levels of the three or more TGFbeta target genes to the activity level of the TGFbeta TF element. In an embodiment the determining of the MAPK-AP1 cellular signaling pathway activity comprises determining an activity level of a MAPK-AP1 transcription factor (TF) element in the sample of the subject, the MAPK-AP1 TF element controlling transcription of the three or more MAPK-AP1 cellular signaling pathway target genes, the determining being based on evaluating a calibrated mathematical pathway model relating the expression levels of the three or more MAPK-AP1 target genes to the activity level of the MAPK-AP1 TF element.

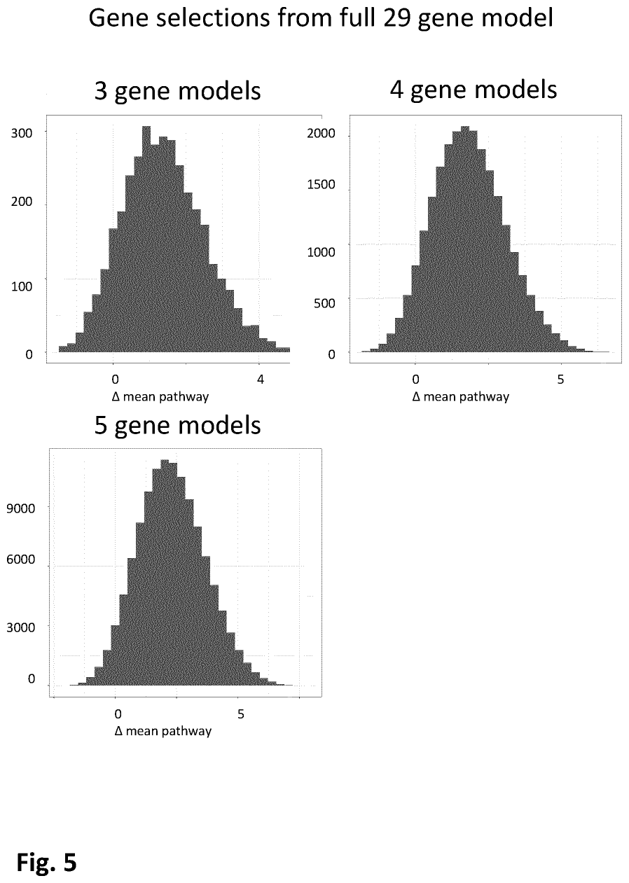

The full model of the TGFbeta pathway activity model described herein comprises 29 genes. The person skilled in the field would appreciate that a pathway activity model using a subset of these genes would still allow to determine pathway activity in an accurate manner and subsequently allow predicting a treatment response. To further support this hypothesis, the inventors have performed additional experiments where subsets of 3, 4 or 5 genes selected from ANGPTL4, CDC42EP3, CDKN1A, CDKN2B, CCN2, GADD45A, GADD45B, HMGA2, ID1, IL11, INPP5D, JUNB, MMP2, MMP9, NKX2-5, OVOL1, PDGFB, PTHLH, SERPINE1, SGK1, SKIL, SMAD4, SMAD5, SMAD6, SMAD7, SNAI1, SNAI2, TIMP1, and VEGFA are used to determine the TGFbeta pathway activity and subsequent prediction or monitoring of a treatment response of a subject with cancer to immunotherapy. These results are described in Example 2 and corresponding FIGS. 5-8. These data support that a selection of three genes selected from ANGPTL4, CDC42EP3, CDKN1A, CDKN2B, CCN2, GADD45A, GADD45B, HMGA2, ID1, IL11, INPP5D, JUNB, MMP2, MMP9, NKX2-5, OVOL1, PDGFB, PTHLH, SERPINE1, SGK1, SKIL, SMAD4, SMAD5, SMAD6, SMAD7, SNAI1, SNAI2, TIMP1, and VEGFA suffice to predict treatment response. Although increasing the number of genes used in the method will improve its accuracy, keeping the number of genes as low as possible may have an advantage as reducing the number of genes tested reduces chances of technical error, cost and complexity of the method. Therefore in a particularly preferred embodiment the invention relates to a method of predicting or monitoring a treatment response of a subject with cancer to immunotherapy, wherein the method is based on a sample obtained from the subject, the method comprising: determining the TGFbeta cellular signaling pathway activity in the sample, wherein an increased TGFbeta cellular signaling pathway activity correlates with a decreased probability of a favorable response to immunotherapy by the subject and wherein a decreased TGFbeta cellular signaling pathway activity correlates with an increased probability of a favorable response to immunotherapy by the subject, making a prediction about the treatment response based on the TGFbeta cellular signaling pathway activity, and wherein the TGFbeta cellular signaling pathway activity is determined based on the expression levels of three or more, preferably four or more or five or more TGFbeta cellular signaling pathway target genes selected from: ANGPTL4, CDC42EP3, CDKN1A, CDKN2B, CCN2, GADD45A, GADD45B, HMGA2, ID1, IL11, INPP5D, JUNB, MMP2, MMP9, NKX2-5, OVOL1, PDGFB, PTHLH, SERPINE1, SGK1, SKIL, SMAD4, SMAD5, SMAD6, SMAD7, SNAI1, SNAI2, TIMP1, and VEGFA, wherein the immunotherapy is an immune checkpoint inhibitor. The immune checkpoint inhibitor may be an immune checkpoint inhibitor aiming to block PD-1, such as an inhibitor of PD-1 or PD-L1.

Further the inventors found that selecting the three or more target genes from a subset of the TGFbeta target genes, ANGPTL4, CDKN1A, CTGF, GADD45A, GADD45B, ID1, IL11, JUNB, MMP2, MMP9, PDGFB, SERPINE1, SGK1, SKIL, SMAD4, SMAD7, SNAI1, TIMP1, and VEGFA, preferably three or more TGFbeta cellular signaling pathway target genes selected from: ANGPTL4, CTGF, IL11, JUNB, MMP2, SERPINE1, SGK1, SKIL, SMAD7, and VEGFA, further improves the accuracy of the prediction, Therefore in a further preferred embodiment the invention relates to a method of predicting or monitoring a treatment response of a subject with cancer to immunotherapy, wherein the method is based on a sample obtained from the subject, the method comprising: determining the TGFbeta cellular signaling pathway activity in the sample, wherein an increased TGFbeta cellular signaling pathway activity correlates with a decreased probability of a favorable response to immunotherapy by the subject and wherein a decreased TGFbeta cellular signaling pathway activity correlates with an increased probability of a favorable response to immunotherapy by the subject, making a prediction about the treatment response based on the TGFbeta cellular signaling pathway activity, and wherein the TGFbeta cellular signaling pathway activity is determined based on the expression levels of three or more, preferably four or more or five or more, such as three, four, five, six, seven, eight, nine or all ten, TGFbeta cellular signaling pathway target genes selected from: ANGPTL4, CDKN1A, CTGF, GADD45A, GADD45B, ID1, IL11, JUNB, MMP2, MMP9, PDGFB, SERPINE1, SGK1, SKIL, SMAD4, SMAD7, SNAI1, TIMP1, and VEGFA, preferably three or more TGFbeta cellular signaling pathway target genes selected from: ANGPTL4, CTGF, IL11, JUNB, MMP2, SERPINE1, SGK1, SKIL, SMAD7, and VEGFA, wherein the immunotherapy is an immune checkpoint inhibitor.

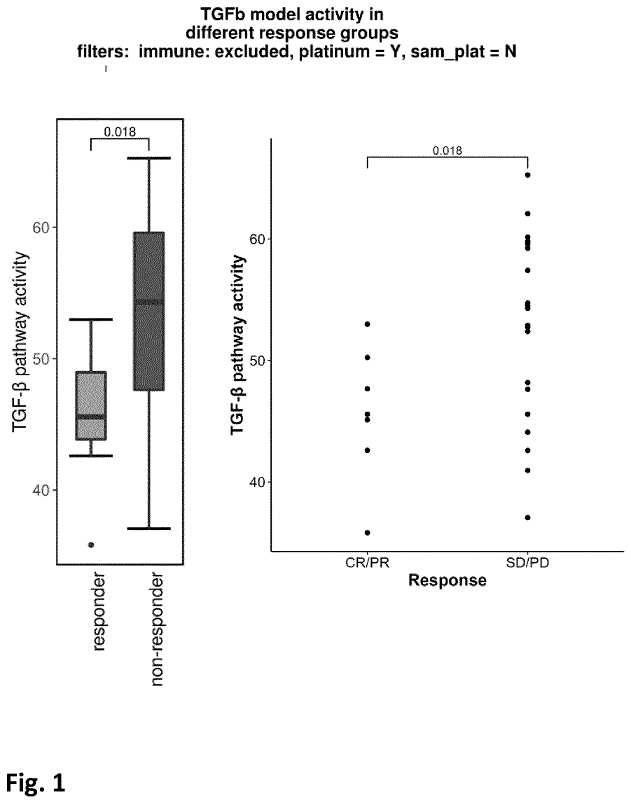

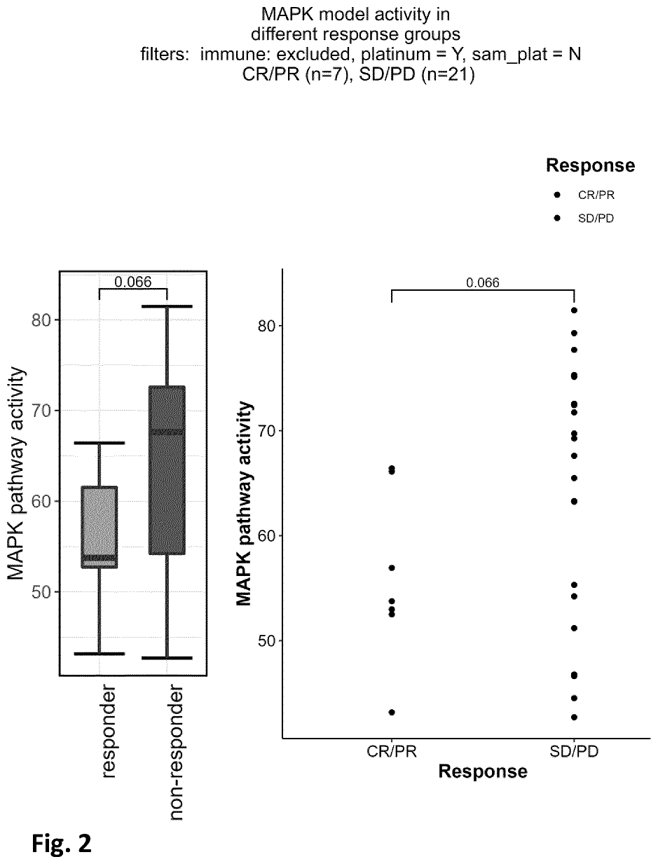

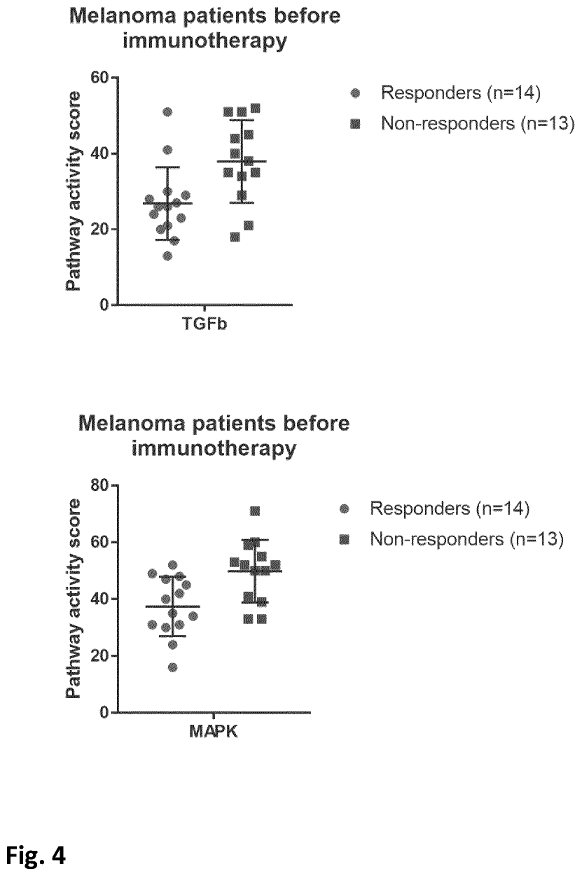

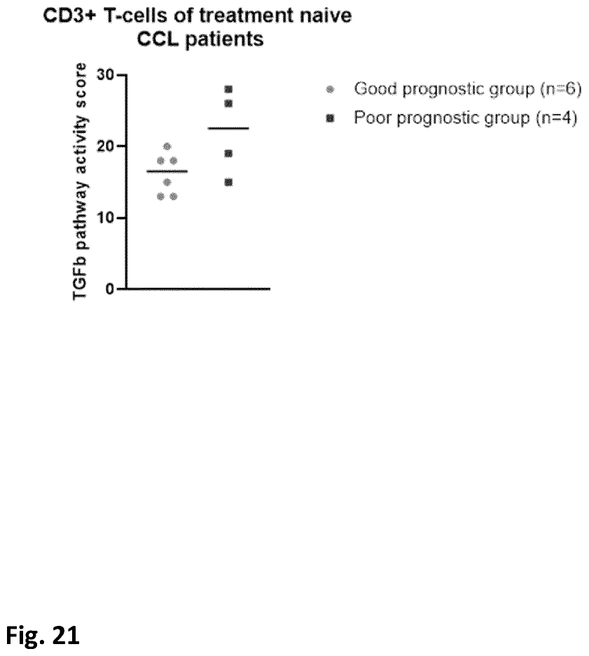

The inventors herein show data from metastatic urothelial cancer (bladder cancer; depicted in FIGS. 1 and 2) and metastatic melanoma (depicted in FIG. 4) and T cells from chronic lymphocytic leukemia patients (FIG. 21). For all tumor types responders and non-responders could be separated accurately based on either TGFbeta or MAPK pathway activity. These are three quite distinct tumor types, however the underlying treatment principle is the same, namely blocking the interaction of tumor expressed PD-L1 with T-cell expressed PD-1. Therefore it is deemed rendered plausible that the method described herein can be used to predict a treatment response to immune checkpoint inhibitors for any tumor type. This holds particularly true for a response to an inhibitor of PD-1 (such as a PD-1 or PD-L1 inhibitor).

When used herein the term immunotherapy should be interpreted as any method or treatment where the immune system of the subject is assisted in targeting one or more tumor cells in said subject. Non limiting examples are immune checkpoint inhibitors, cancer vaccines, CAR-T-cell transfer therapy, monoclonal antibody therapy, immune system modulators, anti TGFbeta therapy and Bacillus Calmette-Guerin (BCG) therapy. Therefore in an embodiment the immunotherapy is selected from: immune checkpoint inhibitors, CAR T-cell transfer therapy, monoclonal antibody therapy, cancer vaccines, immune system modulators and BCG therapy or anti TGFbeta therapy. In the method according to the invention the immunotherapy is an immune checkpoint inhibitor. The immune checkpoint inhibitor may be selected from an inhibitor of PD-1, PD-L1, CTLA-4, CD80, CD86, LAG-3, MHC-II, TIM-3, Galectin9, TIGIT, CD122, CD155, ICOS, ICOS-L, OX-40, OX-40L, or CISH, more preferably an antibody targeting PD-1, PD-L1, CTLA-4, CD80, CD86, LAG-3, MHC-II, TIM-3, Galectin9, TIGIT, CD122, CD155, ICOS, ICOS-L, OX-40, or OX-40L. Suitable antibody or small molecule inhibitors for these targets are known to the skilled person. Non limiting examples are: Ipilimumab (Yervoy), tremelimumab, Nivolumab (Opdivo), Pembrolizumab (Keytruda), Atezolizumab (Tecentriq), Avelumab (Bavencio), Durvalumab (Imfinzi), Cemiplimab (Libtayo), Dostarlimab (Jemperli), JTX-4014, Spartalizumab (PDR001), Camrelizumab (SHR1210), Sintilimab (IBI308), Tislelizumab (BGB-A317), Toripalimab (JS 001), Relatlimab (BMS-986016), INCMGA00012 (MGA012), AMP-224, AMP-514 (MEDI0680), KN035, CK-301, AUNP12, CA-170, BCD-100, and BMS-986189, even more preferably an antibody treatment selected from Ipilimumab (Yervoy), tremelimumab, Nivolumab (Opdivo), Pembrolizumab (Keytruda), Atezolizumab (Tecentriq), Avelumab (Bavencio), Durvalumab (Imfinzi), Cemiplimab (Libtayo), Dostarlimab (Jemperli), JTX-4014, Spartalizumab (PDR001), Camrelizumab (SHR1210), Sintilimab (IBI308), Tislelizumab (BGB-A317), Toripalimab (JS 001), INCMGA00012 (MGA012), AMP-224, AMP-514 (MEDI0680), KN035, CK-301, AUNP12, CA-170, LY3300054, INCAGN02385, Sym023, Cobolimab (TSR-022), RO7121661, AZD7789, LY3321367, LB1410, INCAGN02390 and BMS-986189.

In a particularly preferred embodiment the immune checkpoint inhibitors are aimed at blocking PD-1. Examples of such immune checkpoint inhibitors are inhibitors of PD-1 or PD-L1. The inventors herein show that for both PD-1 inhibitors as PD-L1 inhibitors a treatment response can be predicted, for example FIGS. 1 and 2 depict data obtained from responders and non-responders to anti PD-L1 treatment while FIG. 4 depicts data obtained from responders and non-responders to anti PD-1 treatment. Non limiting examples of PD-1 inhibitors are: Pembrolizumab (formerly MK-3475 or lambrolizumab, Keytruda), Nivolumab (Opdivo), Cemiplimab (Libtayo), Dostarlimab (Jemperli), Retifanlimab (Zynyz), Vopratelimab (JTX-4014), Spartalizumab (PDR001), Camrelizumab (SHR1210) Sintilimab (IBI308), Tislelizumab (BGB-A317), Toripalimab (JS 001), INCMGA00012 (MGA012), AMP-224, AMP-514 (MEDI0680) and Acrixolimab (YBL-006). All of these examples except AMP-224, AMP-514 (MEDI0680) and Acrixolimab (YBL-006) are antibody based inhibitors, the latter being small molecule based inhibitors. Non limiting examples of PD-L1 inhibitors are Atezolizumab (Tecentriq), Avelumab (Bavencio), Durvalumab (Imfinzi), KN035, Cosibelimab (CK-301), AUNP12, CA-170, and BMS-986189. All of these examples except AUNP12, CA-170, and BMS-986189 are antibody based inhibitors, AUNP12 is a 29-mer peptide and CA-170, and BMS-986189 are small molecule based inhibitors.

Accordingly in an embodiment the method predicts or monitors a treatment response to an immune checkpoint inhibitor selected from Pembrolizumab (formerly MK-3475 or lambrolizumab, Keytruda), Nivolumab (Opdivo), Cemiplimab (Libtayo), Dostarlimab (Jemperli), Retifanlimab (Zynyz), Vopratelimab (JTX-4014), Spartalizumab (PDR001), Camrelizumab (SHR1210) Sintilimab (IBI308), Tislelizumab (BGB-A317), Toripalimab (JS 001), INCMGA00012 (MGA012), AMP-224, AMP-514 (MEDI0680), Acrixolimab (YBL-006), Atezolizumab (Tecentriq), Avelumab (Bavencio), Durvalumab (Imfinzi), KN035, Cosibelimab (CK-301), AUNP12, CA-170, and BMS-986189.

In a further aspect the invention relates to the use of an immunotherapy in the treatment or amelioration of cancer, wherein the immunotherapy is administered if, based on the method described herein, there is a reasonable chance that the therapy will be successful. Therefore, in an aspect the invention relates to an immunotherapy for use in the treatment of cancer in a subject in need thereof, wherein the use comprises: determining the TGFbeta cellular signaling pathway activity and/or the MAPK-AP1 cellular signaling pathway activity in the sample, wherein an increased TGFbeta and/or the MAPK-AP1 cellular signaling pathway activity correlates with a decreased probability of a favorable response to immunotherapy by the subject and wherein a decreased TGFbeta and/or MAPK-AP1 cellular signaling pathway activity correlates with an increased probability of a favorable response to immunotherapy by the subject, making a prediction about the treatment response based on the TGFbeta cellular signaling pathway activity and/or the MAPK-AP1 cellular signaling pathway activity, and wherein the TGFbeta cellular signaling pathway activity is determined based on the expression levels of three or more TGFbeta cellular signaling pathway target genes selected from: ANGPTL4, CDC42EP3, CDKN1A, CDKN2B, CCN2, GADD45A, GADD45B, HMGA2, ID1, IL11, INPP5D, JUNB, MMP2, MMP9, NKX2-5, OVOL1, PDGFB, PTHLH, SERPINE1, SGK1, SKIL, SMAD4, SMAD5, SMAD6, SMAD7, SNAI1, SNAI2, TIMP1, and VEGFA, wherein the MAPK-AP1 cellular signaling pathway activity is determined based on the expression levels of three or more AP1 target genes selected from: BCL2L11, CCND1, DDIT3, DNMT1, EGFR, ENPP2, EZR, FASLG, FIGF, GLRX, IL2, IVL, LOR, MMP1, MMP3, MMP9, SERPINE1, PLAU, PLAUR, PTGS2, SNCG, TIMP1, TP53, and VIM.

Alternatively, the invention relates to a method of treating or ameliorating a subject with cancer, the method comprising: determining the TGFbeta cellular signaling pathway activity and/or the MAPK-AP1 cellular signaling pathway activity in the sample, wherein an increased TGFbeta and/or the MAPK-AP1 cellular signaling pathway activity correlates with a decreased probability of a favorable response to immunotherapy by the subject and wherein a decreased TGFbeta and/or MAPK-AP1 cellular signaling pathway activity correlates with an increased probability of a favorable response to immunotherapy by the subject, making a prediction about the treatment response based on the TGFbeta cellular signaling pathway activity and/or the MAPK-AP1 cellular signaling pathway activity, and wherein the TGFbeta cellular signaling pathway activity is determined based on the expression levels of three or more TGFbeta cellular signaling pathway target genes selected from: ANGPTL4, CDC42EP3, CDKN1A, CDKN2B, CCN2, GADD45A, GADD45B, HMGA2, ID1, IL11, INPP5D, JUNB, MMP2, MMP9, NKX2-5, OVOL1, PDGFB, PTHLH, SERPINE1, SGK1, SKIL, SMAD4, SMAD5, SMAD6, SMAD7, SNAI1, SNAI2, TIMP1, and VEGFA, wherein the MAPK-AP1 cellular signaling pathway activity is determined based on the expression levels of three or more AP1 target genes selected from: BCL2L11, CCND1, DDIT3, DNMT1, EGFR, ENPP2, EZR, FASLG, FIGF, GLRX, IL2, IVL, LOR, MMP1, MMP3, MMP9, SERPINE1, PLAU, PLAUR, PTGS2, SNCG, TIMP1, TP53, and VIM,
and administering to the subject an immunotherapy when the TGFbeta and/or the MAPK cellular signaling pathway activity is low, or administering to the subject an alternative treatment when the TGFbeta and/or the MAPK cellular signaling pathway activity is high. Preferably the immunotherapy is an immune checkpoint inhibitor. Even more preferably an immune checkpoint inhibitor inhibiting PD-1 or PD-L1. For example the immunotherapy can be administered when the TGFbeta and/or MAPK cellular signaling pathway activity is not found aberrantly high or an alternative treatment is administered when the TGFbeta and/or MAPK cellular signaling pathway activity is found aberrantly high. Alternatively, the immunotherapy can be administered when the TGFbeta and/or MAPK cellular signaling pathway activity is found to be below a preset threshold or an alternative treatment is administered when the TGFbeta and/or MAPK cellular signaling pathway activity is found to exceed. Alternatively, the immunotherapy can be administered when the TGFbeta and/or MAPK cellular signaling pathway activity is found to be such that it corresponds with an acceptable probability that the immunotherapy will succeed or an alternative treatment is administered when the TGFbeta and/or MAPK cellular signaling pathway activity is found to be such that it corresponds with an unacceptably low probability that the immunotherapy will succeed.

In an embodiment, the immunotherapy is selected from: immune checkpoint inhibitors, CAR T-cell transfer therapy, monoclonal antibody therapy, cancer vaccines, immune system modulators and BCG therapy or anti-TGFbeta therapy, preferably wherein the immunotherapy is an immune checkpoint inhibitor selected from an inhibitor of PD-1, PD-L1, CTLA-4, CD80, CD86, LAG-3, MHC-II, TIM-3, Galectin9, TIGIT, CD122, CD155, ICOS, ICOS-L, OX-40, OX-40L, or CISH, more preferably an antibody targeting PD-1, PD-L1, CTLA-4, CD80, CD86, LAG-3, MHC-II, TIM-3, Galectin9, TIGIT, CD122, CD155, ICOS, ICOS-L, OX-40, or OX-40L. Non limiting examples of suitable inhibitors are listed herein above. In an embodiment the immune checkpoint inhibitor is selected from Pembrolizumab (formerly MK-3475 or lambrolizumab, Keytruda), Nivolumab (Opdivo), Cemiplimab (Libtayo), Dostarlimab (Jemperli), Retifanlimab (Zynyz), Vopratelimab (JTX-4014), Spartalizumab (PDR001), Camrelizumab (SHR1210) Sintilimab (IBI308), Tislelizumab (BGB-A317), Toripalimab (JS 001), INCMGA00012 (MGA012), AMP-224, AMP-514 (MEDI0680), Acrixolimab (YBL-006), Atezolizumab (Tecentriq), Avelumab (Bavencio), Durvalumab (Imfinzi), KN035, Cosibelimab (CK-301), AUNP12, CA-170, and BMS-986189.

In an embodiment the alternative therapy includes administering immunotherapy combined with an inhibitor of TGFbeta and/or an inhibitor of the MAPK signaling cascade, preferably an inhibitor of the TGFbeta pathway. n an aspect the invention relates to a kit of parts comprising:
means for determining the expression levels of three or more genes selected from: ANGPTL4, CDC42EP3, CDKN1A, CDKN2B, CCN2, GADD45A, GADD45B, HMGA2, ID1, IL11, INPP5D, JUNB, MMP2, MMP9, NKX2-5, OVOL1, PDGFB, PTHLH, SERPINE1, SGK1, SKIL, SMAD4, SMAD5, SMAD6, SMAD7, SNAI1, SNAI2, TIMP1, and VEGFA; and/or
means for determining the expression levels of three or more genes selected from: BCL2L11, CCND1, DDIT3, DNMT1, EGFR, ENPP2, EZR, FASLG, FIGF, GLRX, IL2, IVL, LOR, MMP1, MMP3, MMP9, SERPINE1, PLAU, PLAUR, PTGS2, SNCG, TIMP1, TP53, and VIM. The kit may further optionally comprise an immunotherapy formulation, preferably wherein the immunotherapy formulation is selected from a formulation for: immune checkpoint inhibitor therapy, CAR T-cell transfer therapy, monoclonal antibody therapy, cancer vaccine therapy, immune system modulator therapy, anti TGFbeta therapy and BCG therapy, preferably wherein the immunotherapy is an immune checkpoint inhibitor selected from an inhibitor of PD-1, PD-L1, CTLA-4, CD80, CD86, LAG-3, MHC-II, TIM-3, Galectin9, TIGIT, CD122, CD155, ICOS, ICOS-L, OX-40, OX-40L, or CISH, more preferably an antibody targeting PD-1, PD-L1, CTLA-4, CD80, CD86, LAG-3, MHC-II, TIM-3, Galectin9, TIGIT, CD122, CD155, ICOS, ICOS-L, OX-40, or OX-40L. In an embodiment the immune checkpoint inhibitor is selected from Pembrolizumab (formerly MK-3475 or lambrolizumab, Keytruda), Nivolumab (Opdivo), Cemiplimab (Libtayo), Dostarlimab (Jemperli), Retifanlimab (Zynyz), Vopratelimab (JTX-4014), Spartalizumab (PDR001), Camrelizumab (SHR1210) Sintilimab (IBI308), Tislelizumab (BGB-A317), Toripalimab (JS 001), INCMGA00012 (MGA012), AMP-224, AMP-514 (MEDI0680), Acrixolimab (YBL-006), Atezolizumab (Tecentriq), Avelumab (Bavencio), Durvalumab (Imfinzi), KN035, Cosibelimab (CK-301), AUNP12, CA-170, and BMS-986189.

The means for determining the expression levels of the target genes may be primers and/or probes suitable for any one of PCR, quantitative PCR, digital PCR, RNA sequencing, or targeted RNA sequencing. Thus for example the means may be PCR primers, quantitative PCR primers and probes, digital PCR primers and probes, or capture probes for targeted mRNA sequencing. Preferably the means are PCR amplification primers and optionally probes or mRNA targeted sequencing capture probes. In an aspect the invention relates to the use of a kit of parts in predicting or monitoring a treatment response of a subject with cancer, the kit comprising:
means for determining the expression levels of three or more genes selected from: ANGPTL4, CDC42EP3, CDKN1A, CDKN2B, CCN2, GADD45A, GADD45B, HMGA2, ID1, IL11, INPP5D, JUNB, MMP2, MMP9, NKX2-5, OVOL1, PDGFB, PTHLH, SERPINE1, SGK1, SKIL, SMAD4, SMAD5, SMAD6, SMAD7, SNAI1, SNAI2, TIMP1, and VEGFA, and/or
primers and/or probes for determining the expression levels of three or more genes selected from: BCL2L11, CCND1, DDIT3, DNMT1, EGFR, ENPP2, EZR, FASLG, FIGF, GLRX, IL2, IVL, LOR, MMP1, MMP3, MMP9, SERPINE1, PLAU, PLAUR, PTGS2, SNCG, TIMP1, TP53, and VIM. In an embodiment the use comprises providing a sample obtained from a patient having a tumor. In an embodiment the use further comprises determining the gene expression levels of three or more TGFbeta target genes using the means provided in the kit. In an embodiment the use further comprises inferring the TGFbeta pathway activity based on the three or more gene expression levels. In an embodiment the use further comprises predicting or monitoring a treatment response of a subject with cancer to an immune checkpoint inhibitor. In an embodiment the immune checkpoint inhibitor is aimed at blocking PD-1. In an embodiment the immune checkpoint inhibitor is an inhibitor of PD-1 or PD-L1. In a preferred embodiment the kit is used in any one of the methods described herein according to the first aspect f the invention. Preferably the use is ex vivo or in vitro. Also disclosed herein is the use of a kit of parts in predicting or monitoring a treatment response of a subject with cancer, the kit comprising: means for determining the expression levels of three or more genes selected from: ANGPTL4, CDC42EP3, CDKN1A, CDKN2B, CCN2, GADD45A, GADD45B, HMGA2, ID1, IL11, INPP5D, JUNB, MMP2, MMP9, NKX2-5, OVOL1, PDGFB, PTHLH, SERPINE1, SGK1, SKIL, SMAD4, SMAD5, SMAD6, SMAD7, SNAI1, SNAI2, TIMP1, and VEGFA, and/or means for determining the expression levels of three or more genes selected from: BCL2L11, CCND1, DDIT3, DNMT1, EGFR, ENPP2, EZR, FASLG, FIGF, GLRX, IL2, IVL, LOR, MMP1, MMP3, MMP9, SERPINE1, PLAU, PLAUR, PTGS2, SNCG, TIMP1, TP53, and VIM.

### EXAMPLES

### Example 1

Data from the IMvigor210 phase 2 clinical trial [Mariathasan et al. (Nature. 2018 Feb. 22; 554(7693):544-548)] was used to assess TGFbeta pathway activities and MAPK-AP1 pathway activities in patients with metastatic urothelial cancer (mUC, referred to as bladder cancer in the remaining of this text). The Imvigor210 dataset contains RNA sequencing data obtained from tumor biopsies from patients with bladder cancer prior to treatment with anti PD-L1 immune checkpoint inhibitors. Patients were followed up and scored based in treatment response, where CR=complete recovery, PR=partial recovery, SD=stable disease and PD=progressive disease. Only samples from tumors with the phenotype ‘immune excluded’ were selected for analysis. A further selection was made by excluding samples that were also treated with platinum-based chemotherapy. FIG. 1 shows the TGFbeta signaling pathway activity of the selected samples. A clear distinction can be made between the complete/partial recovery group and the stable/progressive group (P=0.018) (left graph). Individual results are plotted in the right graph.

The results on the same set of samples for the MAPK-AP1 signaling pathway is shown in FIG. 2. A clear distinction can be made between the complete/partial recovery group and the stable/progressive group (P=0.066) (left graph). Individual results are plotted in the right graph.

For the same set of samples the expression levels of TGFbeta receptor TGFbetaR2 were examined and results shown in FIG. 3. No significant difference can be observed between the complete/partial recovery group and the stable/progressive group (P=0.23).

A public dataset from another study (GSE78220, Hugo et al. (Cell 165, 35-44, 2016)) contains RNA sequencing data from tumor biopsies from metastatic melanoma patients, obtained prior to treatment with anti PD-1 immune checkpoint inhibitors. The TGFbeta and MAPK-AP1 signaling pathway activities of these samples were calculated and are shown in FIG. 4.

Statistical analysis was performed using Mann Whitney test (T-test unpaired non-parametric): TGFbeta responders vs non-responders: P=0.0102; MAPK responders vs non-responders: P=0.0045.

### Example 2

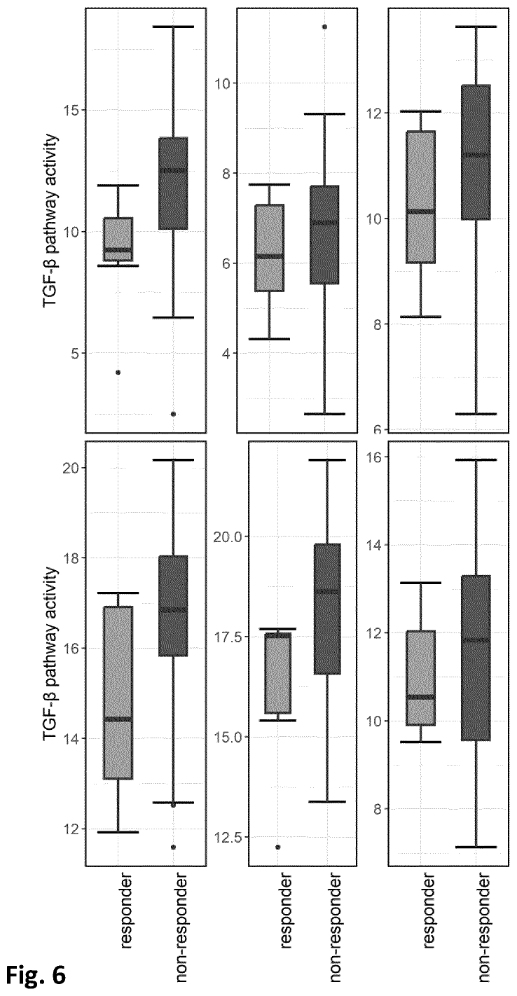

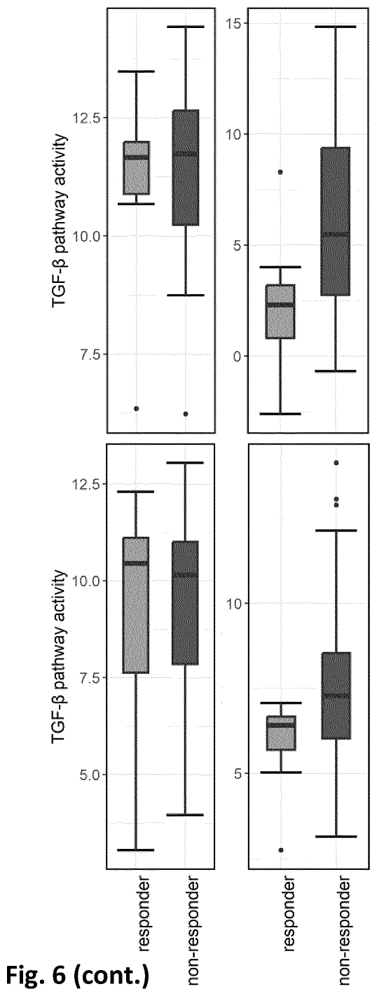

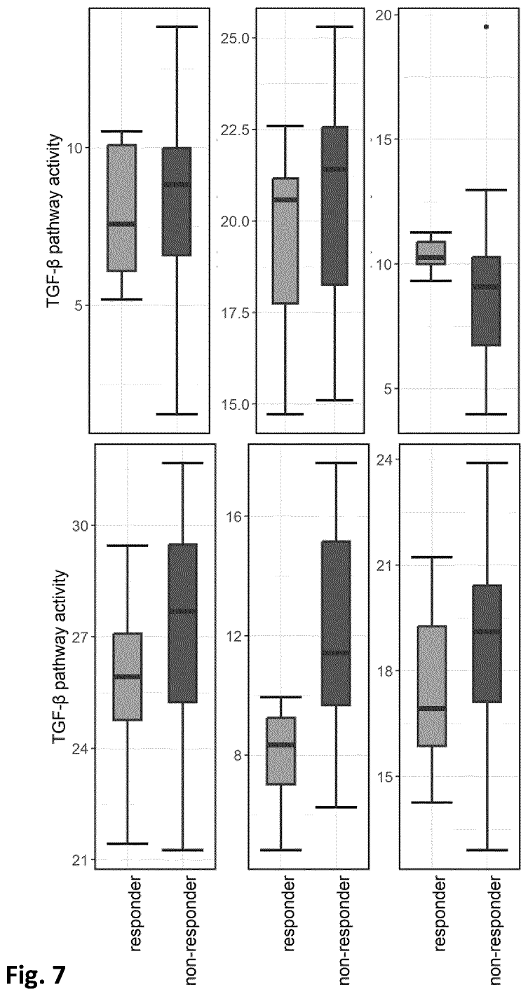

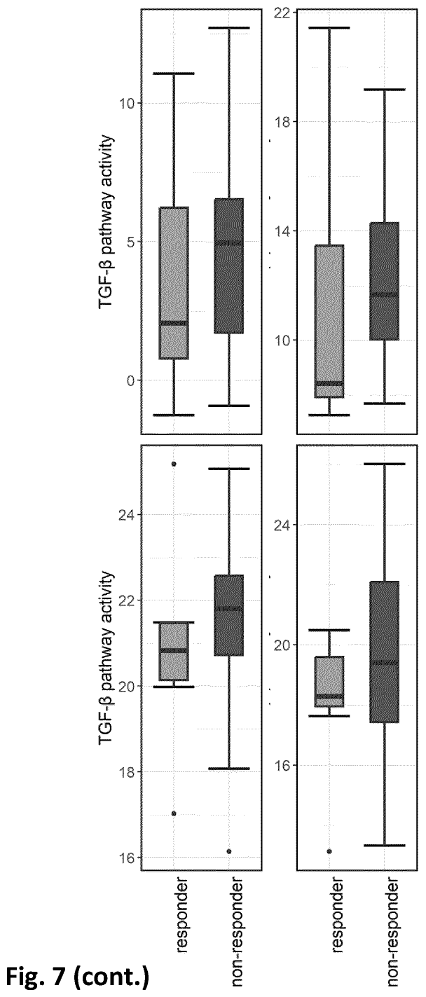

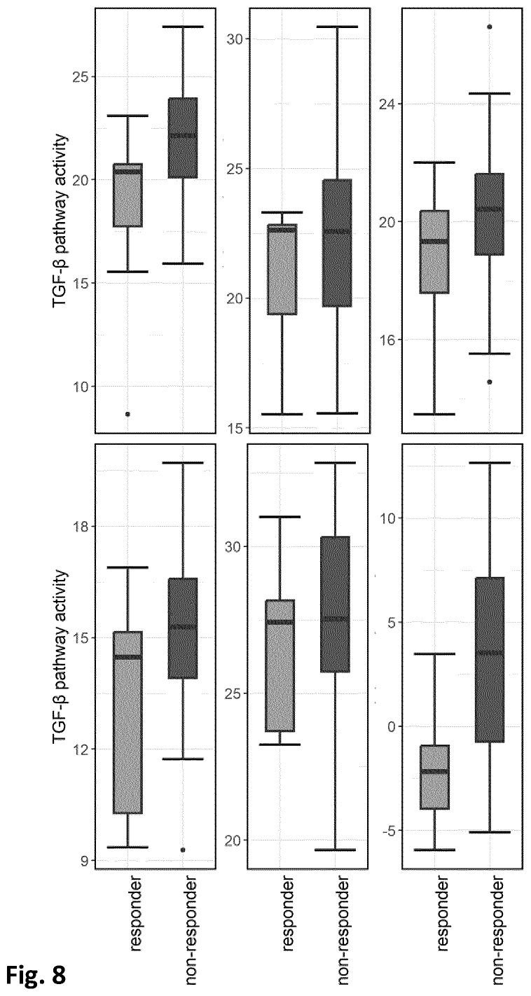

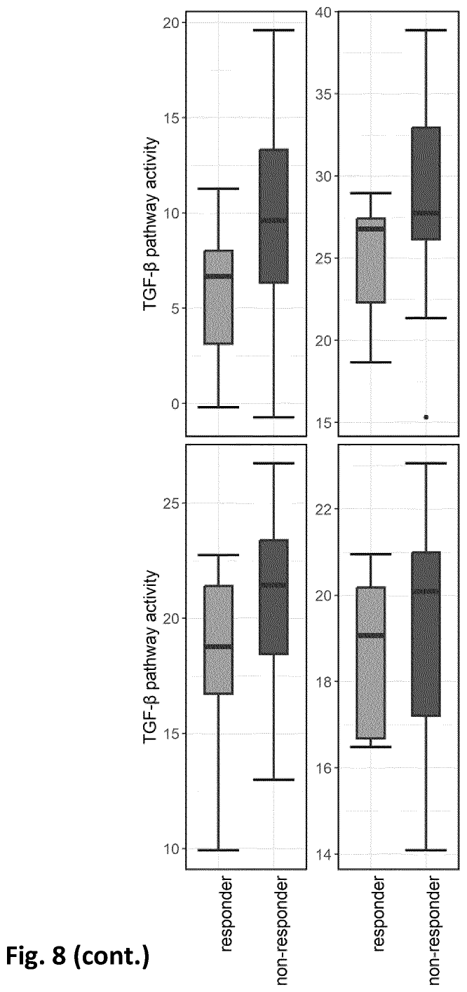

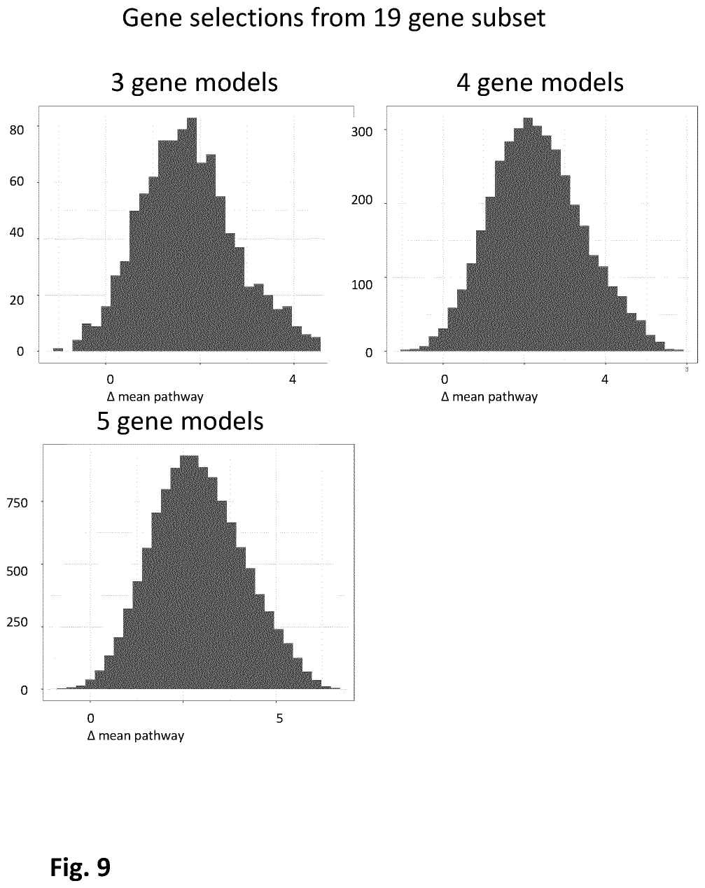

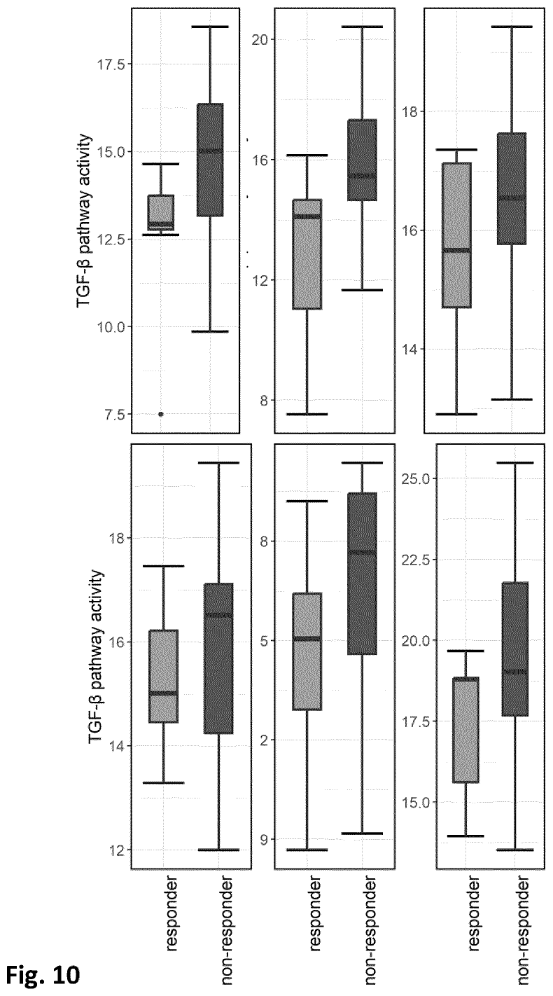

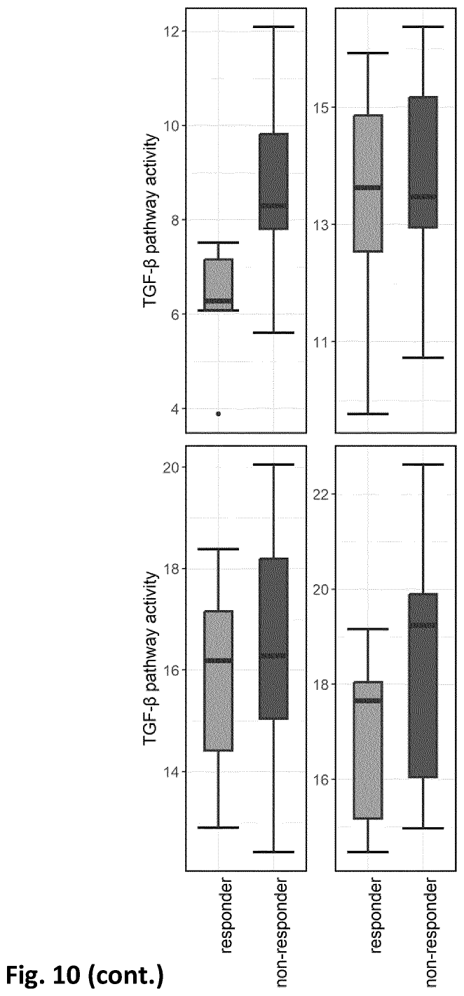

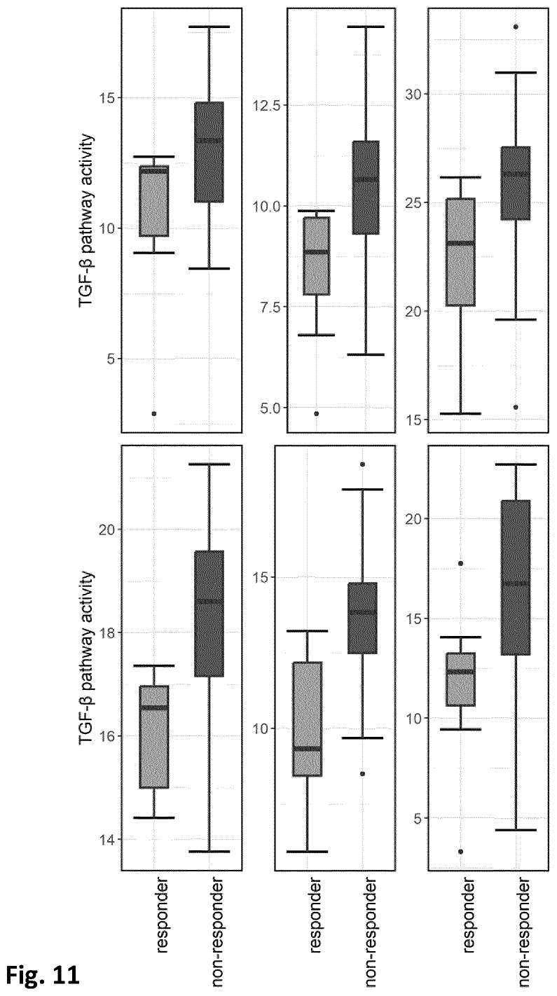

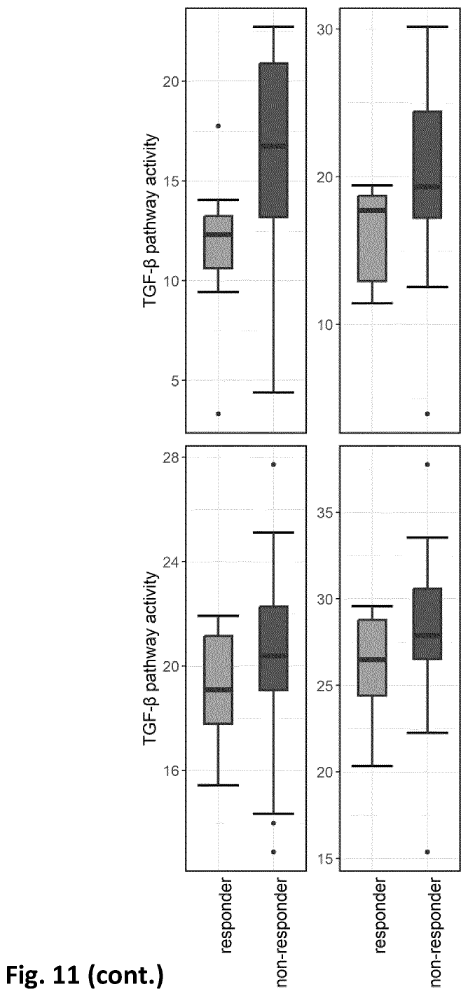

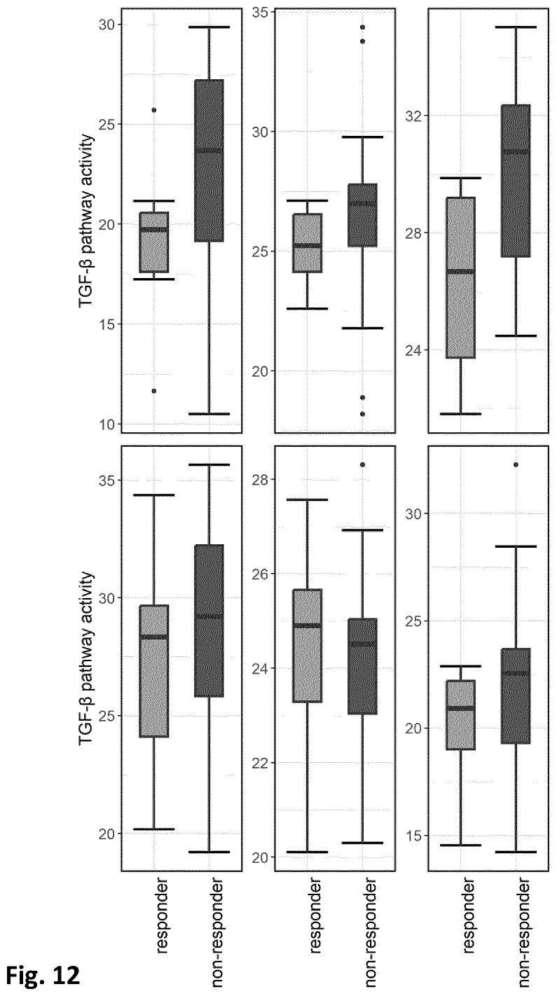

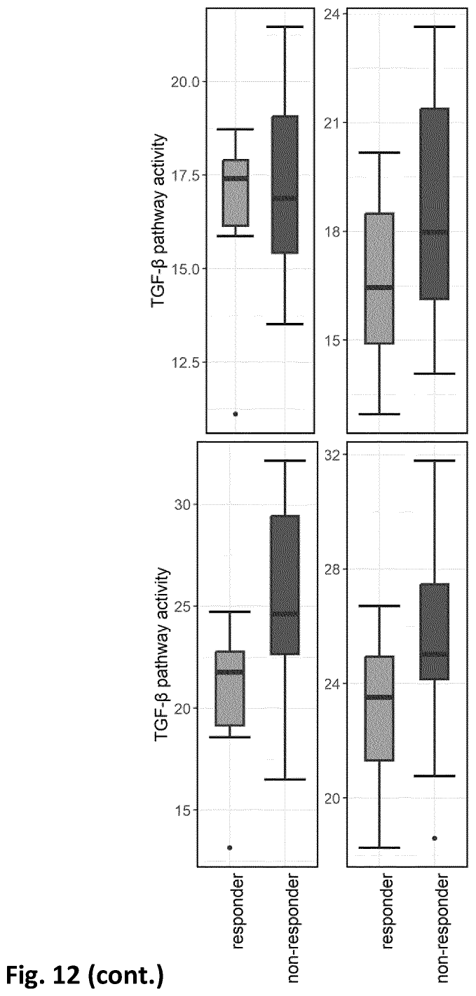

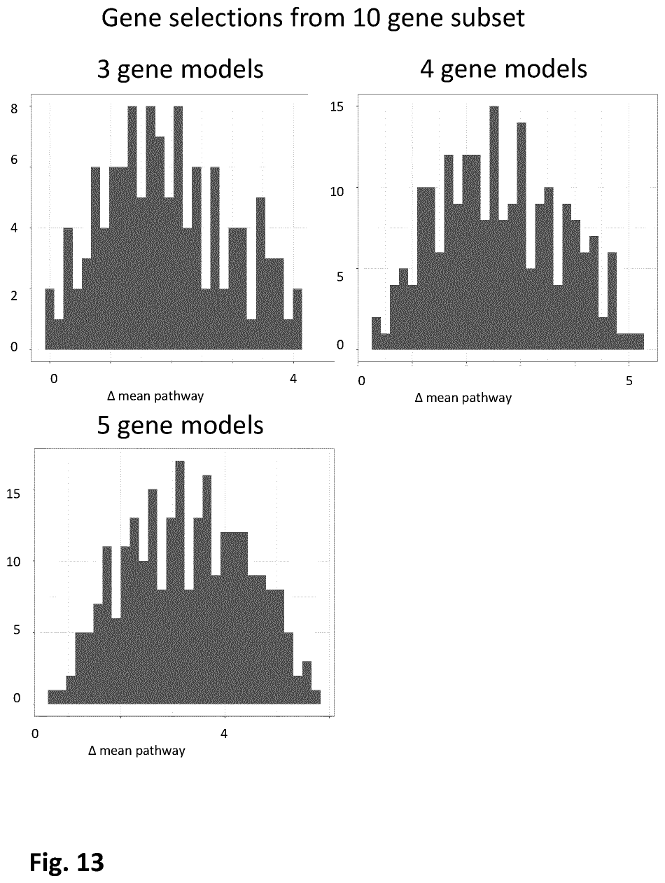

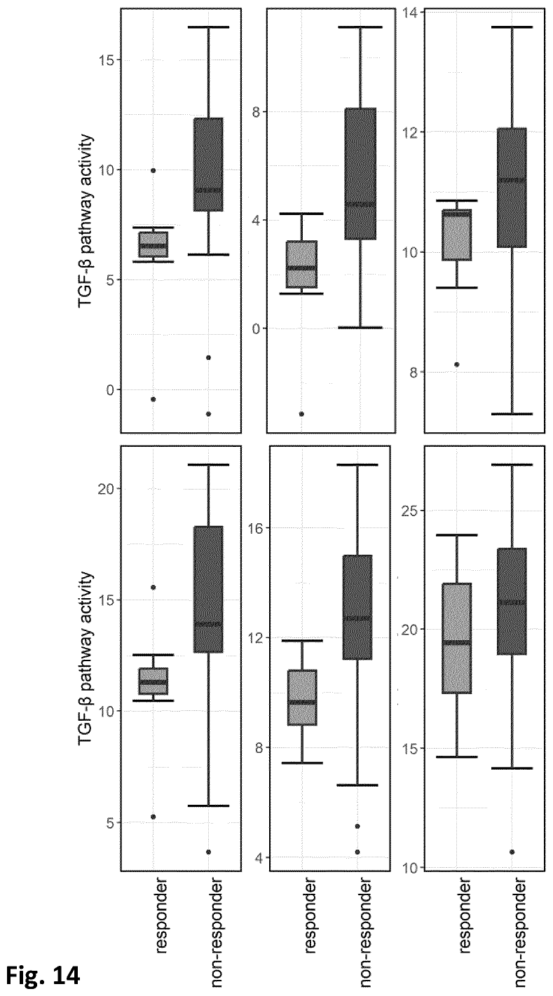

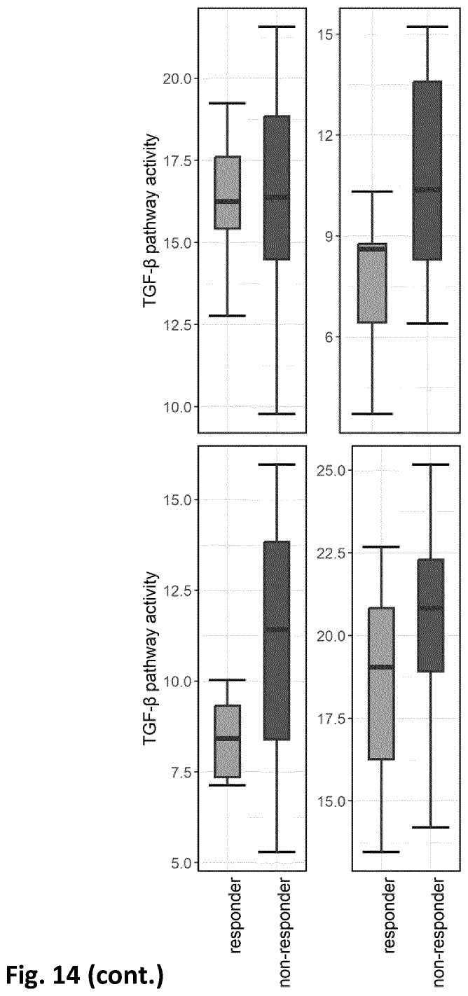

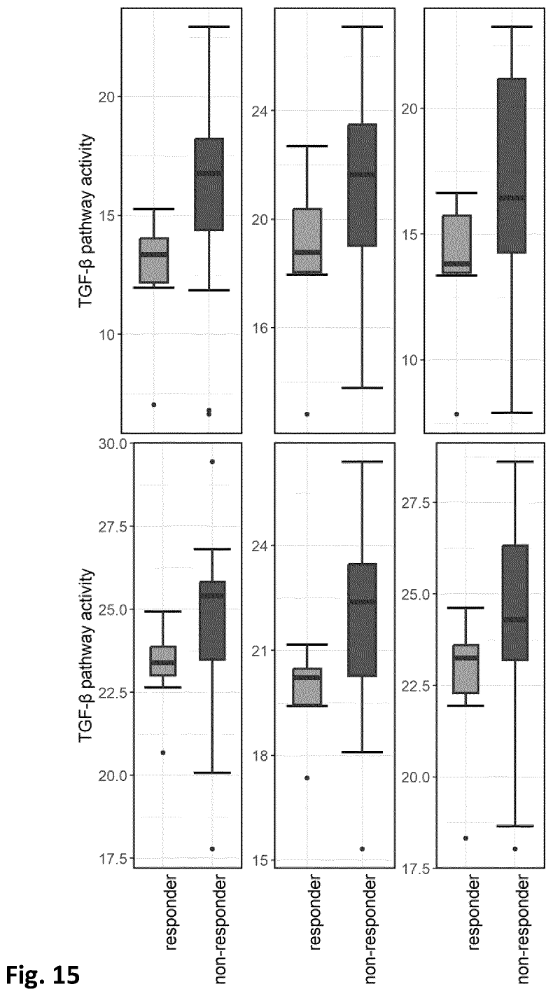

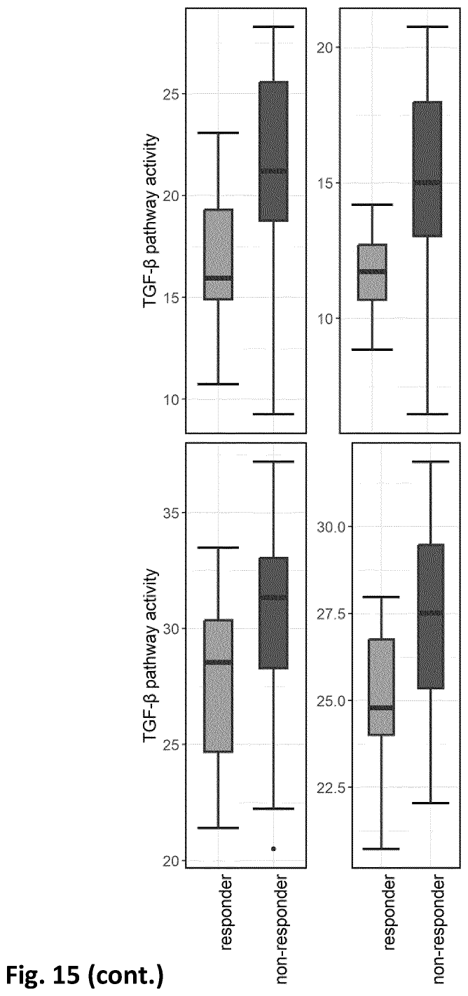

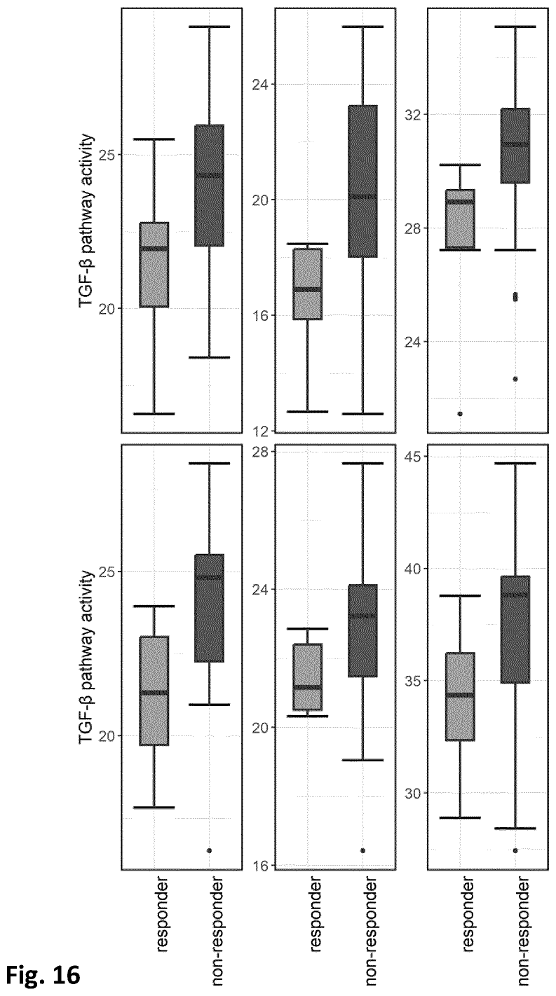

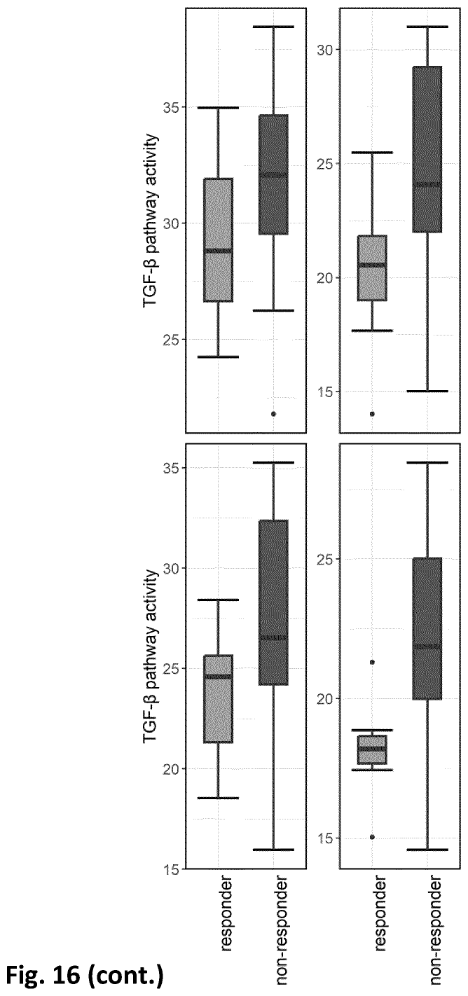

Next the inventors set out to see whether a subset of target genes suffices to predict therapy response. In order to do so all 3, 4 and 5 genes models were generated from the full 29 gene TGFbeta target gene set (FIGS. 5-8), a 19 gene subset of TGFbeta target genes (FIGS. 9-12) or a 10 gene subset of TGFbeta target genes (FIGS. 13-16). First for each 3, 4 or 5 gene pathway model, pathway activity was calculated for the responder and non-responder groups from the IMvigor210 phase 2 clinical trial dataset described above, and mean pathway activities for the response and non-responder group were calculated for each 3, 4, or 5 gene model. Next the difference of the mean pathway was calculated and plotted on a histogram (FIG. 5). AS can be seen the number of models resulting in a vlue of zero or a negative value (meaning the mean pathway activity calculated with the specific model is equal or higher for responders) is negligible for either 3, 4 or 5 gene based pathway models. The inventors concluded that the vast majority of the 3, 4, and 5 gene pathway model are ably to differentiate between responders and non-responders. As an indication 10 3, 4 and 5 gene models were randomly selected as representative examples and box plots are plotted for the results obtained with each of these models in FIGS. 6-8 (3 gene models FIG. 6, 4 gene models FIGS. 7 and 5 gene models FIG. 8). Consist with the above the majority of the different models were capable of differentiating between TGFbeta inferred pathway activity and thus able to predict treatment response based on said inferred activity. Based on these results the inventors concluded that it has been made plausible that a model of 3 genes selected from ANGPTL4, CDC42EP3, CDKN1A, CDKN2B, CCN2, GADD45A, GADD45B, HMGA2, ID1, IL11, INPP5D, JUNB, MMP2, MMP9, NKX2-5, OVOL1, PDGFB, PTHLH, SERPINE1, SGK1, SKIL, SMAD4, SMAD5, SMAD6, SMAD7, SNAI1, SNAI2, TIMP1, and VEGFA suffices to predict a treatment response.

Next the inventors investigated if accuracy of the predictive model can be improved by selecting 3, 4, or 5 genes from smaller subsets of TGFbeta target genes which may be more predictive specifically for immunotherapy treatment response (e.g. immune checkpoint inhibitor treatment response). Therefore two subsets of TGFbeta target genes were generated selected from the full set of TGFbeta target genes:
a) a 19 gene subset comprising the target genes: ANGPTL4, CDKN1A, CTGF, GADD45A, GADD45B, ID1, IL11, JUNB, MMP2, MMP9, PDGFB, SERPINE1, SGK1, SKIL, SMAD4, SMAD7, SNAI1, TIMP1, and VEGFA; and
b) a 10 gene subset comprising the target genes: ANGPTL4, CTGF, IL11, JUNB, MMP2, SERPINE1, SGK1, SKIL, SMAD7, and VEGFA.

The above experiments were repeated but now the 3, 4, and 5 gene models were selected from the 19 gene subset or the 10 gene subset respectively. The data resresented in FIGS. 9-12 was generated by selecting 3, 4, or 5 gene models form the 19 gene subset, the data represented by FIGS. 13-16. As demonstrated particularly by the histograms (FIGS. 9 and 13, when compared to FIG. 5) an improvement can be observed by selecting the target genes form the 19 gene subset and a further improvement by selecting from the 10 gene subset.

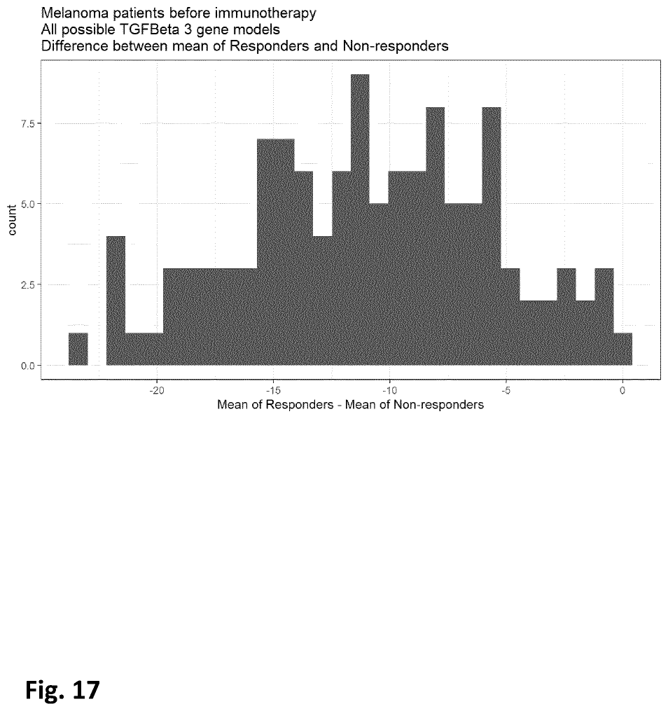

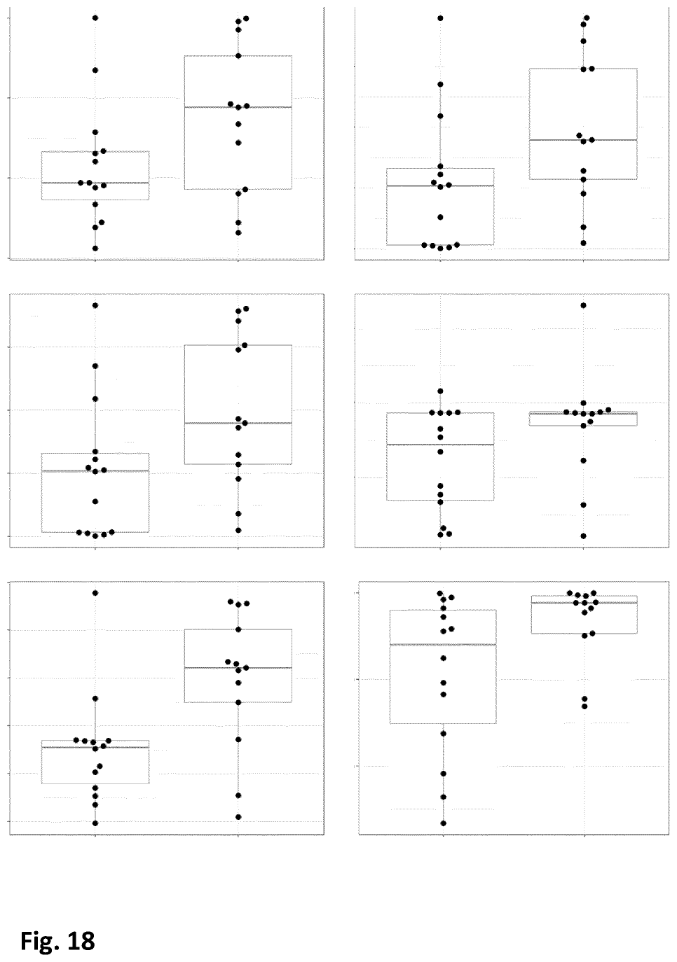

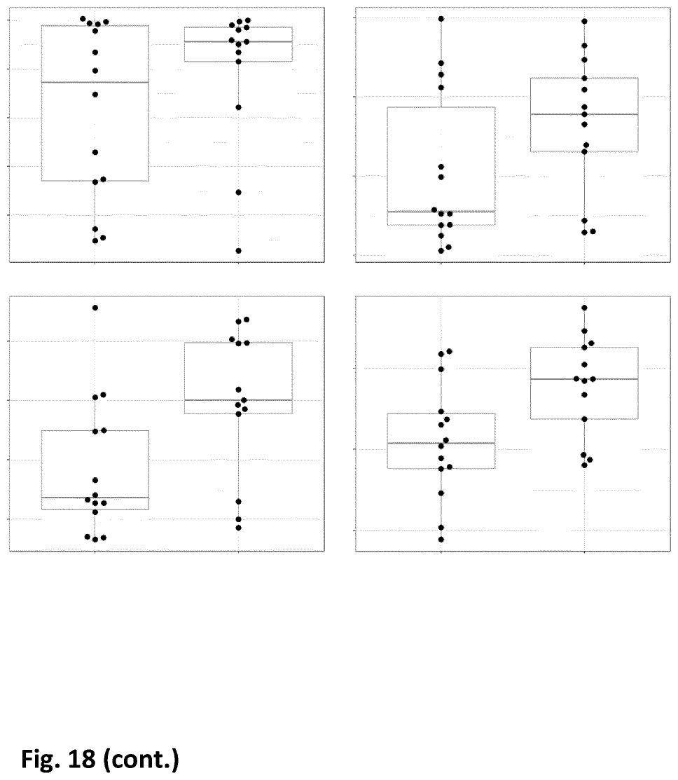

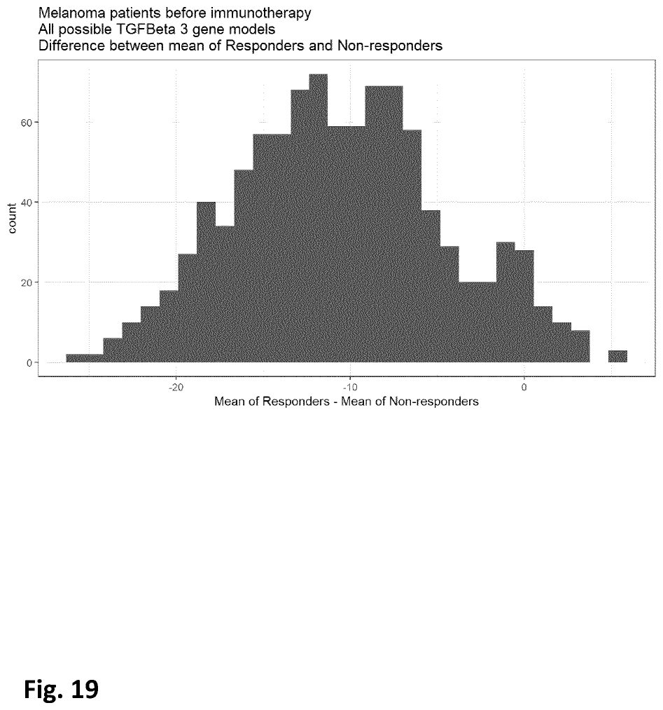

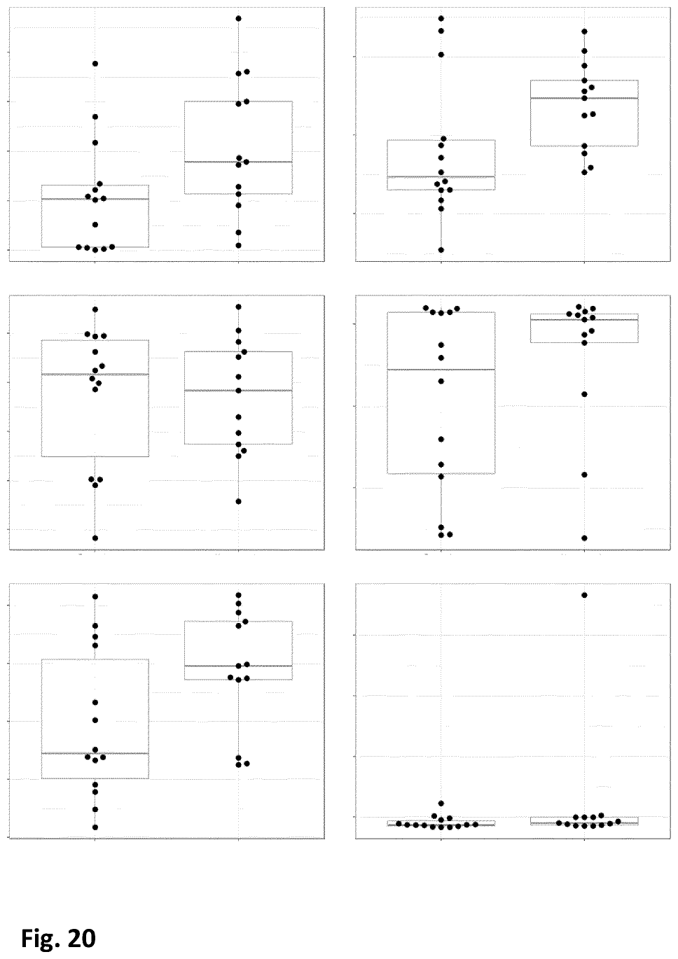

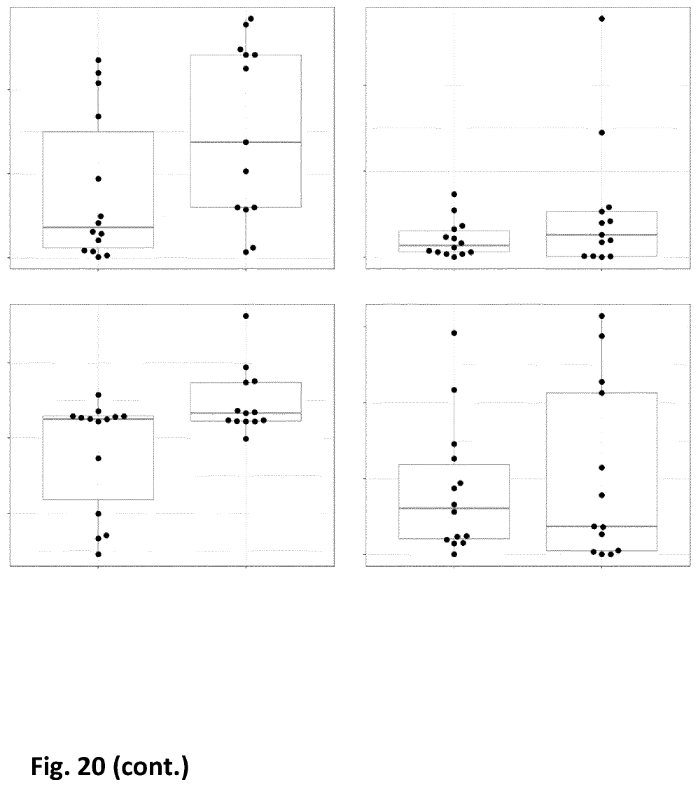

Next the experiment was repeated on the melanoma dataset (GSE78220, described above), using 3, 4, or 5 genes selected from the 10 gene subset (FIGS. 17 and 18) or selected from the 19 gene subset (FIGS. 19 and 20). Again histograms were plotted, note that now the mean non-responder calculated pathway activity was subtracted from the mean responder pathway activity, meaning that a negative value is observed if a difference in pathway activity is measured. Also in this dataset it was confirmed that a selection of 3, 4, or 5 target genes suffices to predict a treatment response in melanoma as well.

To further investigate the general underlying principle of TGFbeta signaling in predicting a treatment response to immunotherapy, an additional dataset was analyzed: GSE148476. This dataset comprises RNA sequencing data obtained from purified T-cells from chronic lymphocytic leukemia (CLL) patients. RNA sequencing was performed on highly purified T cells from treatment naïve CLL patients treated for 18 hours with avadomide or anti-PD-1 (nivolumab) or anti-PD-L1 (durvalumab). alone or in combinations. Patients samples were selected to represent extremes of prognosis (n=6 good prognosis and n=6 poor prognosis). TGFbeta pathway activity was calculated for each sample individually and plotted in FIG. 21. A clear correlation can be observed for TGFbeta pathway activity and treatment response in T cells. The inventors concluded that these data further support the general underlying principle of TGFbeta pathway activity as a predictive factor for immunotherapy treatment response in cancer. Further, without wishing to be bound be theory, these data provide a plausible explanation that measuring TGFbeta pathway activity in T cells present in the sample at least partly results in a general principle of predicting treatment response. The inventors here present that T cells themselves display different responses to immunotherapy (PD-1 or PD-L1 inhibitors) and differences in TGFbeta pathway activity correlate with treatment response. It is theorized that the TGFbeta pathway activity in the T cells at least partially causes the T cells to kill tumor cells in the presence of immunotherapeutic compounds (when activity is low), while a high activity appears to block response to immunotherapy. It is not excluded that TGFbeta pathway activity in the tumor cells themselves serves a similar role.

1. A method of predicting or monitoring a treatment response of a subject with cancer to immunotherapy, wherein the method is based on a sample obtained from the subject, the method comprising:
determining TGFbeta cellular signaling pathway activity in the sample obtained from the subject, wherein an increased TGFbeta cellular signaling pathway activity correlates with a decreased probability of a favorable treatment response to immunotherapy by the subject and wherein a decreased TGFbeta cellular signaling pathway activity correlates with an increased probability of a favorable treatment response to immunotherapy by the subject; and
making a prediction about the subject's treatment response to immunotherapy based on the determined TGFbeta cellular signaling pathway activity in the sample obtained from the subject,
wherein the TGFbeta cellular signaling pathway activity is determined based on the expression levels of three or more TGFbeta cellular signaling pathway target genes selected from: ANGPTL4, CDC42EP3, CDKN1A, CDKN2B, CCN2, GADD45A, GADD45B, HMGA2, ID1, IL11, INPP5D, JUNB, MMP2, MMP9, NKX2-5, OVOL1, PDGFB, PTHLH, SERPINE1, SGK1, SKIL, SMAD4, SMAD5, SMAD6, SMAD7, SNAI1, SNAI2, TIMP1, and VEGFA; and
wherein the immunotherapy is an immune checkpoint inhibitor.

2. The m method of claim 1, wherein the method further comprises comparing the determined TGFbeta cellular signaling pathway activity with a predetermined threshold, and wherein the prediction is based on a comparison of the determined TGFbeta cellular signaling pathway activity with the predetermined threshold.

3. The m method of claim 1, wherein the method comprises:
receiving expression levels of three or more target genes of the TGFbeta cellular signaling pathway measured in the sample of the subject, wherein the method is a computer implemented method, or wherein the method comprises determining expression levels of three or more target genes of the TGFbeta cellular signaling pathway in a sample obtained from the subject.

4. The method of claim 1, wherein the determining the TGFbeta cellular signaling pathway activity comprises determining an activity level of a TGFbeta transcription factor (TF) element in the sample of the subject, the TGFbeta TF element controlling transcription of the three or more TGFbeta cellular signaling pathway target genes, the determining being based on evaluating a calibrated mathematical pathway model relating the expression levels of the three or more TGFbeta target genes to the activity level of the TGFbeta TF element.

5. The m method of claim 1, wherein the immune checkpoint inhibitor is an inhibitor of PD-1 or PD-L1, and wherein the immune checkpoint inhibitor is selected from a group consisting of Pembrolizumab (formerly MK-3475 or lambrolizumab, Keytruda), Nivolumab (Opdivo), Cemiplimab (Libtayo), Dostarlimab (Jemperli), Retifanlimab (Zynyz), Vopratelimab (JTX-4014), Spartalizumab (PDR001), Camrelizumab (SHR1210) Sintilimab (IBI308), Tislelizumab (BGB-A317), Toripalimab (JS 001), INCMGA00012 (MGA012), AMP-224, AMP-514 (MEDI0680), Acrixolimab (YBL-006), Atezolizumab (Tecentriq), Avelumab (Bavencio), Durvalumab (Imfinzi), KN035, Cosibelimab (CK-301), AUNP12, CA-170, and BMS-986189.

6. The method of claim 1, wherein the sample is a tissue sample, a blood sample, a biopsy, or a sample from the subject comprising cells.

7. The method of claim 2, wherein the threshold is predetermined in a set of samples obtained from a cohort of cancer patients.

8. The method of claim 1, wherein the treatment response of the subject is favorable or non-favorable response to the immunotherapy treatment.

9. The method of claim 1, wherein the response is complete response, partial response, stable disease, or disease progression.

10. The method of claim 1, wherein the method further comprises providing a treatment advice to a medical care giver, wherein the treatment advice is to provide the immunotherapy.

11. The method of claim 1, wherein the method is for monitoring a treatment response, wherein the method further comprises providing a continuation advice to a medical care giver, wherein the continuation advice is to continue a current immunotherapy treatment strategy or to change the current immunotherapy treatment strategy.

12. The method of claim 1, wherein the TGFbeta cellular signaling pathway activity is determined based on expression levels of three or more TGFbeta cellular signaling pathway target genes selected from: ANGPTL4, CDKN1A, CTGF, GADD45A, GADD45B, ID1, IL11, JUNB, MMP2, MMP9, PDGFB, SERPINE1, SGK1, SKIL, SMAD4, SMAD7, SNAI1, TIMP1, and VEGFA, or wherein the TGFbeta cellular signaling pathway activity is determined based on expression levels of three or more TGFbeta cellular signaling pathway target genes selected from: ANGPTL4, CTGF, IL11, JUNB, MMP2, SERPINE1, SGK1, SKIL, SMAD7, and VEGFA.

13. A method for using immunotherapy in the treatment of cancer in a subject diagnosed with cancer, comprising:
determining TGFbeta cellular signaling pathway activity in sample obtained from the subject diagnosed with cancer, wherein an increased TGFbeta cellular signaling pathway activity correlates with a decreased probability of a favorable response to immunotherapy by the subject and wherein a decreased TGFbeta cellular signaling pathway activity correlates with an increased probability of a favorable response to immunotherapy by the subject; and
making a prediction about the treatment response based on the determined TGFbeta cellular signaling pathway activity;
wherein the TGFbeta cellular signaling pathway activity is determined based on the expression levels of three or more TGFbeta cellular signaling pathway target genes selected from: ANGPTL4, CDC42EP3, CDKN1A, CDKN2B, CCN2, GADD45A, GADD45B, HMGA2, ID1, IL11, INPP5D, JUNB, MMP2, MMP9, NKX2-5, OVOL1, PDGFB, PTHLH, SERPINE1, SGK1, SKIL, SMAD4, SMAD5, SMAD6, SMAD7, SNAI1, SNAI2, TIMP1, and VEGFA; and
wherein the immunotherapy is an immune checkpoint inhibitor.

14. The method of claim 13, wherein the immune checkpoint inhibitor is an inhibitor of PD-1 or PD-L1, and wherein the immune checkpoint inhibitor is selected from a group consisting of Pembrolizumab (formerly MK-3475 or lambrolizumab, Keytruda), Nivolumab (Opdivo), Cemiplimab (Libtayo), Dostarlimab (Jemperli), Retifanlimab (Zynyz), Vopratelimab (JTX-4014), Spartalizumab (PDR001), Camrelizumab (SHR1210) Sintilimab (IBI308), Tislelizumab (BGB-A317), Toripalimab (JS 001), INCMGA00012 (MGA012), AMP-224, AMP-514 (MEDI0680), Acrixolimab (YBL-006), Atezolizumab (Tecentriq), Avelumab (Bavencio), Durvalumab (Imfinzi), KN035, Cosibelimab (CK-301), AUNP12, CA-170, and BMS-986189.

15. A kit of parts in predicting or monitoring an immunotherapy treatment response of a subject with cancer, the kit comprising:
components for determining expression levels of three or more genes selected from: ANGPTL4, CDC42EP3, CDKN1A, CDKN2B, CCN2, GADD45A, GADD45B, HMGA2, ID1, IL11, INPP5D, JUNB, MMP2, MMP9, NKX2-5, OVOL1, PDGFB, PTHLH, SERPINE1, SGK1, SKIL, SMAD4, SMAD5, SMAD6, SMAD7, SNAI1, SNAI2, TIMP1, and VEGFA.
